(19) United States
(12) Patent Application Publication
(10) Pub. No.: US 2024/0425631 A1
(43) Pub. Date: Dec. 26, 2024

(54) LOW VISCOSITY URETHANE (METH)ACRYLATE MONOMERS AND THEIR USE IN PRODUCTION OF TOUGH POLYMERS WITH WELL-CONTROLLED MODULUS AND STRENGTH

(71) Applicants: The Regents of the University of Colorado, a body corporate, Denver, CO (US); Hybrid Ceramic, LLC, San Francisco, CA (US)

(72) Inventors: Jeffrey W. STANSBURY, Centennial, CO (US); Austyn SALAZAR, Denver, CO (US); Matthew D. BARROS, Denver, CO (US); Steven J. SADOWSKY, San Francisco, CA (US)

(21) Appl. No.: 18/706,650

(22) PCT Filed: Nov. 1, 2022
(86) PCT No.: PCT/US2022/079064

§ 371 Date: May 1, 2024

Related U.S. Application Data
(60) Provisional application No. 63/274,348, filed on Nov. 1, 2021.

(51) Int. Cl. C08F 220/34; A61K 6/887; C08F 222/10; C08J 5/18

(52) U.S. Cl. CPC C08F 220/343; C08F 222/1065; C08J 5/18; A61K 6/887

(57) ABSTRACT

Low viscosity urethane (meth)acrylate monomers and polymerizable resin compositions are provided to facilitate production of tough polymers with well-controlled modulus and strength.

### CROSS-REFERENCES TO RELATED APPLICATIONS

This application is being filed on Nov. 1, 2022, as a PCT International application, and claims priority to and the benefit of U.S. provisional application No. 63/274,348, filed Nov. 1, 2021, the entire contents of which are incorporated herein by reference.

### STATEMENT REGARDING FEDERALLY SPONSORED RESEARCH OR DEVELOPMENT

This invention was made with government support under grant number R21 DE028444 awarded by National Institutes of Health/National Institute of Dental and Craniofacial Research NIH/NIDCR. The government has certain rights in the invention.

### BACKGROUND

Urethane (meth)acrylates are one of the most widely used class of radically cured monomers and oligomers that find application in coatings, binders, adhesives, sealants, printing inks, elastomers, flexible packaging, and dental/biomedical materials.

Urethane (meth)acrylates offer good reactivity and mechanical properties as (co) polymers that can be varied from highly extensible rubbery polymers to glassy solid networks that can combine good strength with what has previously been considered to be good toughness. These reactive materials are typically made by reacting a di- or multi-isocyanate with a hydroxy functionalized (meth)acrylate such as 2-hydroxyethyl(meth)acrylate [HE(M)A] to produce monomers, or by the reaction of a diol or polyol with an excess of diisocyanate with the oligomeric product then end-capped with HE(M)A or a related hydroxy-substituted (meth)acrylate. The extended hydrogen bonding of the multi-urethane groups produces relatively high viscosities that are typically in the 10³-10⁵ mPa·s range, or even solids, that require addition of reactive diluent comonomers to render the resins readily processable in the pre-cure state; however, this approach also dilutes the urethane group concentration that is key for the hydrogen bonding reinforcement that contributes toward the desirable properties of urethane (meth)acrylate polymers. This may lead to characteristic additive property behavior based on the proportions of the urethane (meth)acrylate and the reactive diluent comonomer. Adding a lower viscosity non-urethane comonomer as a reactive diluent not only reduces the resin viscosity to practically functional levels, but it also dilutes the overall urethane content of the resin, which typically reduces both the polymeric strength and toughness that are the desirable properties conveyed by the urethane-based monomers.

Improved urethane (meth)acrylate polymerizable compositions having low viscosity amenable to 3D printing, while exhibiting improved polymerization conversion, greater strength, and increased toughness, and that are suitable for use in dental applications, or other applications, are desirable.

### SUMMARY

Low viscosity, polymerizable resin compositions are provided that are useful to facilitate production of copolymers with well-controlled modulus and strength that meet or exceed current best properties while also simultaneously providing uniquely high toughness.

Polymerizable resin compositions are provided comprising urethane (meth)acrylate monomers and acidic comonomers, wherein a stoichiometric excess of the acidic comonomer acidic functionality relative to the urethane functional groups increases the high-performance character, which is unexpected. This differs from the conventional use of a diluent comonomer to control the viscosity of a urethane-based resin since the addition of a conventional diluent monomer in any significant amount tends to reduce the ultimate mechanical strength and toughness of the polymer by diluting the urethane functionality that is typically responsible for the good properties of polyurethanes and urethane (meth)acrylates.

The disclosure provides a low viscosity polymerizable resin composition comprising a urethane (meth)acrylate monomer, and a comonomer selected from the group consisting of an acidic comonomer, an anhydride comonomer, and a monourethane mono(meth)acrylate comonomer, wherein the urethane (meth)acrylate monomer is selected from the group consisting of a multi-urethane (meth)acrylate monomer and a monourethane di(meth)acrylate comonomer.

The disclosure provides a low viscosity polymerizable resin composition comprising a multi-urethane (meth)acrylate monomer, and a comonomer or comonomers selected from the group consisting of an acidic comonomer, an anhydride comonomer, a monourethane mono(meth)acrylate comonomer, and a monourethane di(meth)acrylate comonomer.

The low viscosity polymerizable resin may comprise a mono-urethane and/or multi-urethane (meth)acrylate monomer and a carboxylic functional or otherwise acidic comonomer. The low viscosity polymerizable resin may comprise a multi-urethane (meth)acrylate monomer and a monourethane mono(meth)acrylate comonomer. The low viscosity polymerizable resin may comprise a multi-urethane (meth)acrylate monomer and a monourethane di(meth)acrylate comonomer.

The low viscosity polymerizable resin composition may have a viscosity at room temperature of no more than 1,000 mPa·s, no more than 500 mPa·s, no more than 300 mPa·s, 100 mPa·s, no more than 80 mPa·s, no more than 60 mPa·s, no more than 40 mPa·s, or no more than 20 mPa·s.

The low viscosity polymerizable resin composition may include a multi-urethane (meth)acrylate monomer that is a linear or branched multi-urethane multi (meth)acrylate. In some embodiments, the low viscosity polymerizable resin composition may include a multi-urethane (meth)acrylate selected from the group consisting of a tetraurethane di(meth)acrylate (TUD(M)A) monomer, a diurethane (meth)acrylate (DUM(M)A) monomer, hexaurethane tri(meth)acrylate (HUT(M)A) monomer, an octaurethane tetra(meth)acrylate (OUT(M)A) monomer, and a diurethane di(meth)acrylate monomer.

The low viscosity polymerizable resin composition may have a ratio of the acidic moieties from the acidic monomer to the urethane moieties from the multi-urethane (meth)acrylate monomer of an acidic moiety: urethane moiety ratio in a range of from about 1:1 to about 12:1, about 2:1 to about 8:1; or >1:1, about 2:1; about 3:1; about 4:1; about 5:1; about 6:1; about 7:1; about 8:1; about 9:1; or about 10:1.

The multi-urethane (meth)acrylate monomer may be a TUD(M)A monomer comprising a chemical structure according to Formula (I):

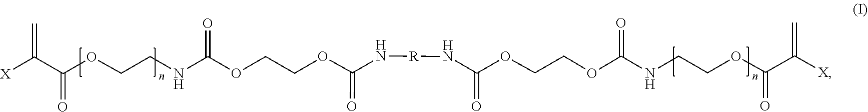

wherein X is —H or —CH₃; n is 1, 2, 3 or 4; and R is aliphatic, alkoxyalkyl, or alkylarylalkyl core group. Optionally, the TUD(M)A of formula (I) may comprise wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group. The TUD(M)A of formula (I) may comprise wherein n is 1 or 2.

In some embodiments, the TUD(M)A of formula (I) may comprise wherein R is a core radical selected from the group consisting of

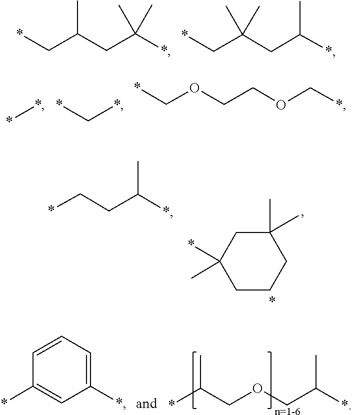

The multi-urethane (meth)acrylate monomer may be a DUM(M)A monomer comprising a chemical structure according to Formula (IV):

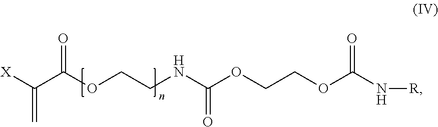

wherein X is —H or —CH₃; n is 1, 2, 3 or 4; and R is aliphatic, alkoxyalkyl, or alkylarylalkyl core group. Optionally, the DUM(M)A of formula (IV) may comprise wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

The multi-urethane (meth)acrylate monomer may be a HUT(M)A monomer comprising a chemical structure according to Formula (V):

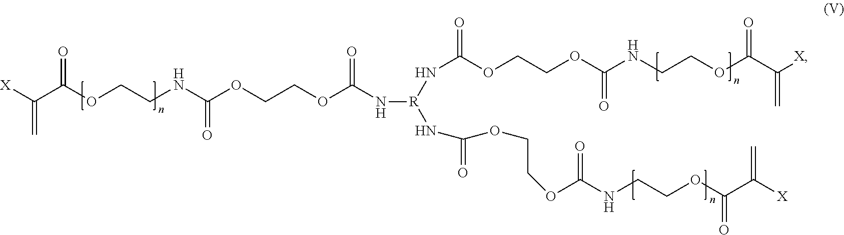

wherein X is —H or —CH₃; n is 1, 2, 3 or 4; and R is aliphatic, alkoxyalkyl, or alkylarylalkyl core group. Optionally, the HUT(M)A of formula (V) may comprise wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

The multi-urethane (meth)acrylate monomer may be an OUT(M)A monomer comprising a chemical structure according to Formula (VI):

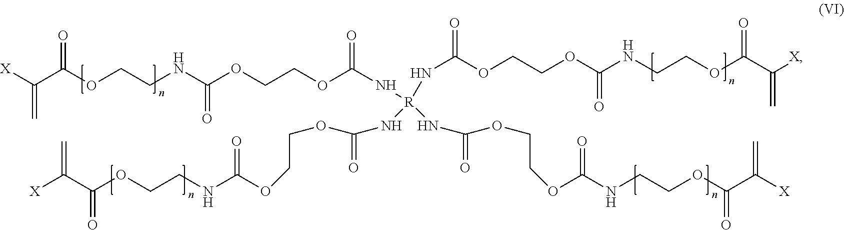

wherein X is —H or —CH₃; n is 1, 2, 3 or 4; and R is aliphatic, alkoxyalkyl, or alkylarylalkyl core group. Optionally, the OUT(M)A of formula (VI) may comprise wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

In some embodiments, the multi-urethane (meth)acrylate monomer may be a diurethane di(meth)acrylate monomer selected from the group consisting of UD(M)A, IPDI-HE(M)A, a PCL diurethane di(meth)acrylate, HE(M)A-MDI, HE(M)A-IPDI, HE(M)A-TDI, HE(M)A-HMDI, HE(M)A-TMXDI, and HE(M)A-XDI.

The disclosure provides a low viscosity polymerizable resin composition comprising a diurethane di(meth)acrylate monomer and an acidic monomer.

The disclosure provides a low viscosity polymerizable resin composition comprising a tetraurethane di(meth)acrylate (TUD(M)A) monomer and an acidic monomer.

The disclosure provides a low viscosity polymerizable resin composition comprising a diurethane (meth)acrylate (DUM(M)A) monomer and an acidic monomer.

The disclosure provides a low viscosity polymerizable resin composition comprising a hexaurethane tri(meth)acrylate (HUT(M)A) monomer and an acidic monomer.

The disclosure provides a low viscosity polymerizable resin composition comprising an octaurethane tetra(meth)acrylate (OUT(M)A) monomer and an acidic monomer.

The acidic comonomer may be a compound comprising one, two, one or more, or two or more acidic moieties and one, two, one or more, or two or more (meth)acrylate moieties. The acidic comonomer may be a compound comprising one acidic moiety and one (meth)acrylate moiety. The acid moiety may comprise a carboxylic acid, carboxylate, or phosphate moiety. The (meth)acrylate moiety may be a methacrylate moiety or an acrylate moiety. The acidic comonomer may be selected from the group consisting of acrylic acid, methacrylic acid (MAA), itaconic acid, mono-2-(methacryloyloxy)ethyl maleate, pyromellitic dianhydride glycerol dimethacrylate, 2-carboxyethyl acrylate, 2-carboxyethyl acrylate oligomer, mono-2-(methacryloyloxy)ethyl succinate, glycerol dimethacrylate/succinate adduct, 1,3-glycerol dimethacrylate/maleate adduct, bis[2-(methacryloyloxy)ethyl]phosphate, or ethylene glycol methacrylate phosphate.

A low viscosity polymerizable resin composition is provided comprising a diurethane di(meth)acrylate monomer, a monourethane mono(meth)acrylate comonomer or a monourethane di(meth)acrylate comonomer; and an acidic comonomer. The molar ratio of the diurethane di(meth)acrylate monomer to the monourethane mono(meth)acrylate monomer or the monourethane di(meth)acrylate monomer is from about 90:10 to about 50:50; about 80:20 to about 50:50; about 70:30, or about 50:50. The ratio of the acidic moieties from the acidic monomer to the urethane moieties from the combined urethane (meth)acrylate monomers are in an acidic moiety: urethane moiety ratio in a range of from about 1:1 to about 10:1, about 2:1 to about 8:1; or >1:1, about 2:1; about 3:1; about 4:1; about 5:1; about 6:1; about 7:1; about 8:1; about 9:1; or about 10:1.

The low viscosity polymerizable resin composition may comprise a monourethane mono(meth)acrylate monomer compound according to Formula (II):

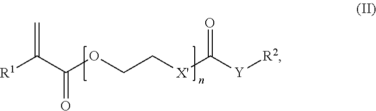

wherein n=1 or 2; R¹ is —H or —CH₃; X′ is O or N; Y is N when X′=O, or Y is O when X′═N; R² is aliphatic, benzyl, alkoxyaryl, alkoxyalkyl, polyalkoxyalkyl, or optionally alkyl C₁₋₁₀ substituted polyalkoxyaryl. The monourethane mono(meth)acrylate monomer may be selected from the group consisting of:

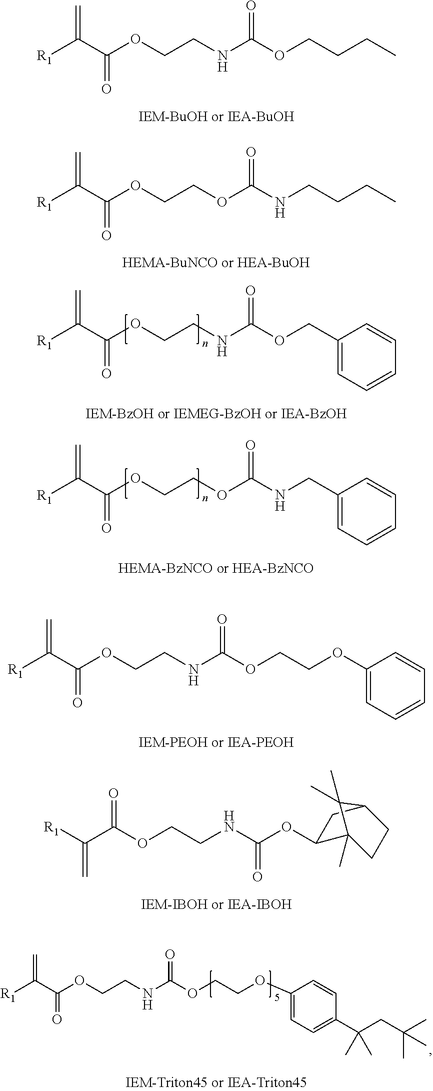

wherein R₁=H, or CH₃; n=1, 2, 3, 4, 5; n=1 for the IEM-BzOH monomer; n=2 for IEMEG-BzOH.

The low viscosity polymerizable resin composition may comprise a monourethane di(meth)acrylate comonomer is a compound according to Formula (IIIa):

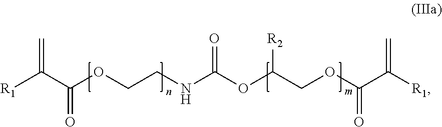

wherein R₁=—H, —CH₃; n-1 or 2; m=1-5; and R₂ is —H, aliphatic, aryl, or alkylaryl group; optionally wherein R₂=H, Me, Et, nPr, iPr, nBu, sBu, tBu, Phe, or Bzl.

In some embodiments, the monourethane di(meth)acrylate comonomer may be selected from the group consisting of:

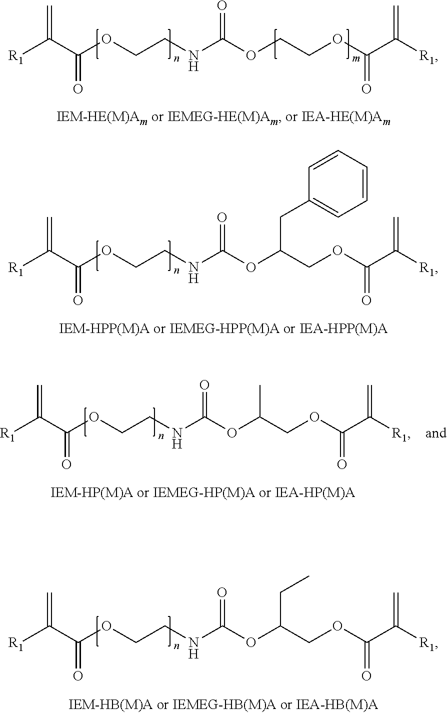

wherein R₁=CH₃ and n=1 for the IEM monomers, R₁=CH₃ and n=2 for the IEMEG monomers, and R₁=H and n=1 for the IEA monomers.

The low viscosity polymerizable resin composition may comprise a mono-urethane di(meth)acrylate comonomer that is a compound according to Formula (IIIb):

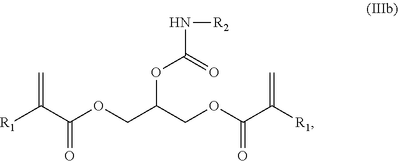

wherein R¹=—H, —CH₃; n=1 or 2; m=1-5, R²=aliphatic, aryl, alkylaryl; optionally wherein R₂=Me, Et, nPr, iPr, nBu, sBu, tBu, Phe, or Bzl.

In some embodiments, the mono-urethane di(meth)acrylate monomer may be selected from the group consisting of

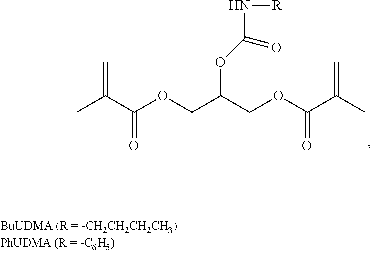

The low viscosity polymerizable resin composition may further comprise an initiator, optionally wherein the initiator is selected from the group consisting of a photoinitiator, a thermal initiator, and a redox initiator.

A polymer prepared from a polymerizable resin according to the disclosure may exhibit: flexural strength of at least 100 MPa, at least 130 MPa, at least 150 MPa, at least 180 MPa, or at least 200 MPa; and flexural modulus of at least 2 GPa, at least 3 GPa, at least 4 GPa, or at least 5 GPa.

A method is provided for preparing a polymer from the low viscosity polymerizable composition according to the disclosure comprising curing the polymerizable resin to form the polymer.

In embodiments, the curing may comprise exposing the polymerizable resin to a condition for a period of time, wherein the condition is selected from the group consisting of light (photocure), elevated temperature above ambient temperature (thermal cure), or redox conditions (redox cure); optionally wherein the light is selected from the group consisting of visible light and ultraviolet light. The photocuring may be used to produce spatial and temporal on-demand polymer formation. The photocuring may be performed within a range of from about 0.5 seconds to about 90 sec; about 1 sec to about 60 sec, about 5 sec to about 30 sec; or about 0.5 sec to about 15 sec; about 0.5 sec to about 10 sec; or about 0.5 sec to about 5 sec, optionally at room temperature. The thermal curing may be performed for a period of time within a range of about 5 sec to about 30 minutes; 10 seconds to 25 minutes; 30 seconds to 20 minutes; 1 minute to 20 minutes; 2 minutes to 15 minutes; or about 10 minutes.

In some embodiments, the polymer exhibits conversion at ambient temperature of at least 60%, or at least 70%, as determined by static or dynamic infrared spectroscopic methods or by another suitable analytical technique.

In some embodiments, the method further comprises post-curing the polymer. The polymer may exhibit post-curing conversion of at least 75%, at least 80%, at least 85%, or at least 90%. Any suitable method for determining post-curing conversion may be employed, for example, by mid infrared spectroscopy, or near infrared spectroscopy, or any other suitable technique.

A method is provided for creating a two-dimensional film or a three-dimensional shaped part comprising molding, free-form fabricating, or printing of the low viscosity polymerizable resin composition according to the disclosure. The method may comprise three-dimensional (3D) printing of the polymerizable resin composition to form a partially or fully cured shaped part; and optionally subjecting to additional post-cure processing to complete production of the shaped part. The shaped part may be a shaped dental appliance, dental prosthetic device, biomedical device, automotive part, microelectronics part, aerospace part, plumbing part, or electrical part.

The disclosure provides a shaped part comprising a polymer created from the polymerization of the polymerizable resin composition according to the disclosure, optionally in admixture with one or more fillers.

The disclosure provides a polymerizable dental appliance or prosthetic material comprising the polymerizable resin composition of the disclosure, and optionally fillers and/or pigments. The polymerizable dental appliance or prosthetic material may comprise a filler in a range of from 0-90 wt %, 0-50 wt %, or 0-25 wt % of the total material weight, and optionally a pigment in a range of from about 0.0001-5 wt %, 0.001-1 wt %, or 0.003-0.5 wt % of the total material weight.

The disclosure provides a dispensing device comprising an unpolymerized quantity of the polymerizable dental appliance or prosthetic material according to the disclosure.

The disclosure provides a tetraurethane di(meth)acrylate (TUD(M)A) monomer compound according to Formula (I)

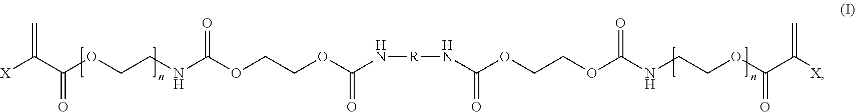

wherein X is —H or —CH₃; n is 1, 2, 3 or 4; and R is aliphatic, alkoxyalkyl, or alkylarylalkyl core group. The TUD(M)A compound according to formula (I) may comprise wherein X is —H or —CH₃; n is 1 or 2; and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group. The TUD(M)A compound according to formula (I) may comprise wherein R is a core radical selected from the group consisting of

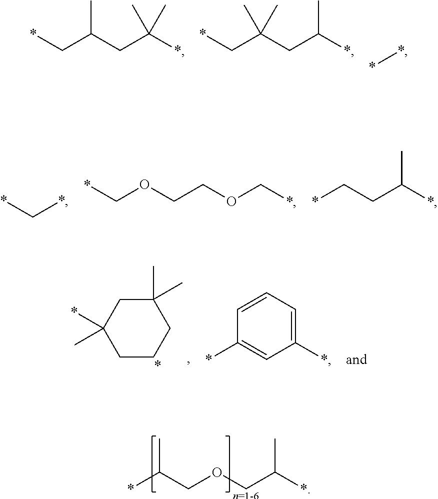

The disclosure provides a monourethane mono(meth)acrylate monomer compound is provided selected from the group consisting of:

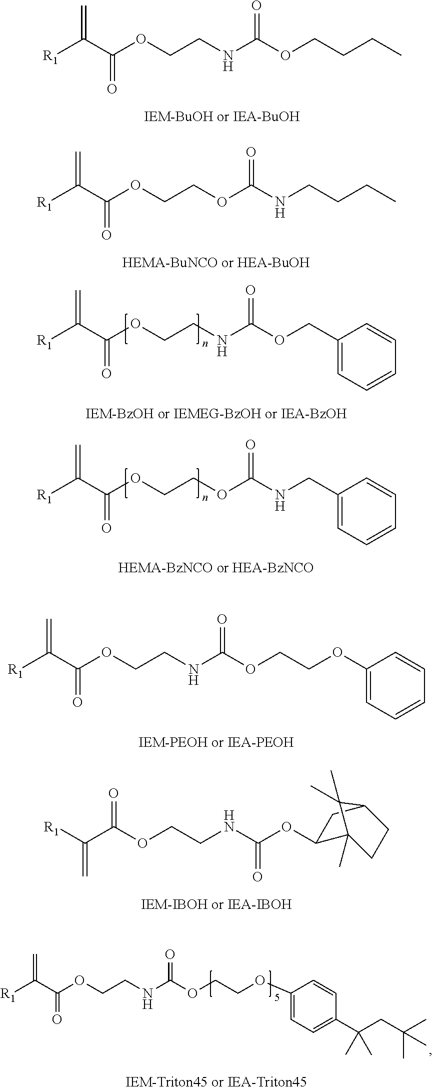

wherein n=1 for the IEM-BzOH monomer, and n=2 for IEMEG-BzOH.

The disclosure provides a mono-urethane di(meth)acrylate monomer compound according to Formula (IIIa):

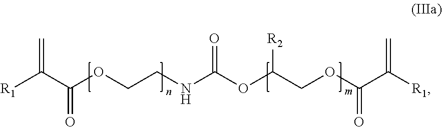

wherein R₁=—H, —CH₃; n=1 or 2; m=1-5; and R₂ is —H, aliphatic, aryl, or alkylaryl group; optionally wherein R₂=H, Me, Et, nPr, iPr, nBu, sBu, tBu, Phe, or Bzl.

The disclosure provides a mono-urethane di(meth)acrylate monomer compound selected from the group consisting of from the group consisting of:

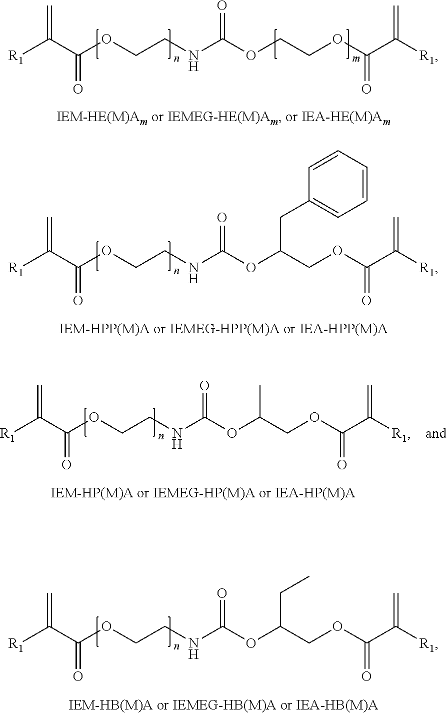

wherein R₁=CH₃ and n=1 for the IEM monomers, R₁=CH₃ and n=2 for the IEMEG monomers, and R₁=H and n=1 for the IEA monomers.

A mono-urethane di(meth)acrylate monomer compound is provided according to Formula (IIIb):

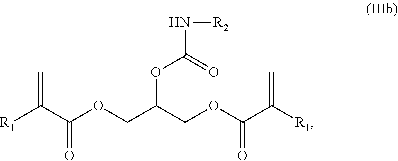

wherein R₁=—H, —CH₃; n=1 or 2; m=1-5, R₂=aliphatic, aryl, alkylaryl; optionally wherein R₂-Me, Et, nPr, iPr, nBu, sBu, tBu, Phe, or Bzl. The mono-urethane di(meth)acrylate monomer compound may be selected from the group consisting of

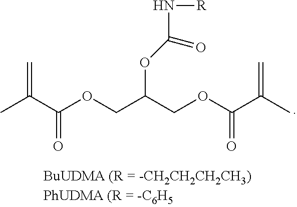

The disclosure provides a diurethane (meth)acrylate (DUM(M)A) monomer compound is provided according to Formula (IV):

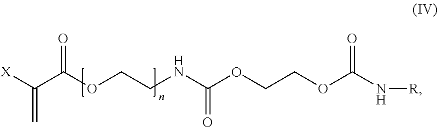

wherein X is —H or —CH₃; n is 1, 2, 3 or 4; and R is aliphatic, alkoxyalkyl, or alkylarylalkyl core group. The DUM(M)A monomer compound may include wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

The disclosure provides a hexaurethane tri(meth)acrylate (HUT(M)A) monomer compound is provided comprising a chemical structure according to Formula (V):

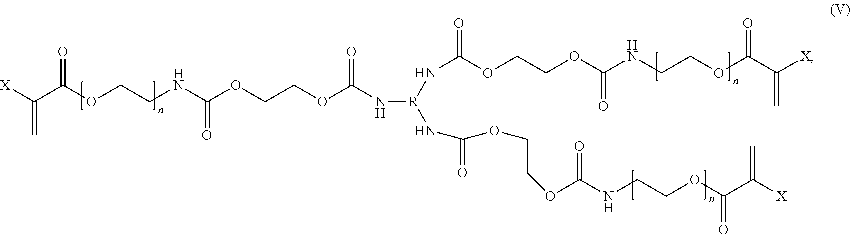

wherein X is —H or —CH₃; n is 1, 2, 3 or 4; and R is aliphatic, alkoxyalkyl, or alkylarylalkyl core group. The HUT(M)A monomer compound may include wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

The disclosure provides an octaurethane tetra(meth)acrylate (OUT(M)A) monomer compound is provided comprising a chemical structure according to Formula (VI):

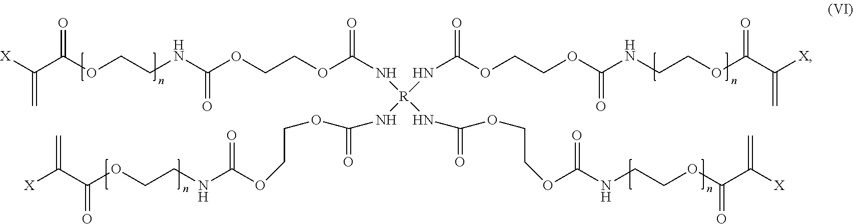

wherein X is —H or —CH₃; n is 1, 2, 3 or 4; and R is aliphatic, alkoxyalkyl, or alkylarylalkyl core group. The OUT(M)A monomer compound may include wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

### BRIEF DESCRIPTION OF THE DRAWINGS

FIG. 1A shows chemical structures of tetra-urethane di(meth)acrylate [TUD(M)A] according to Formula (I) with various core structures provided by different diamine R groups (1-7) as well as variations in the methacrylate (X=—CH₃) or acrylate (X=—H) functionality, and in the spacer group length (n=1 or 2) between the reactive group and the external urethane group.

FIG. 1B shows chemical structures and generic synthetic schemes for synthesis of representative diurethane (meth)acrylate [DUM(M)A]monomers, tetraurethane di(meth)acrylate [TUD(M)A)]monomers, hexaurethane tri(meth)acrylate [HUT(M)A]monomers, and octaurethane tetra(meth)acrylate [OUT(M)A]monomers.

FIG. 2 shows chemical structures of commercially available diurethane dimethacrylate monomers that may be prepared from trimethylhexyl diisocyanate or isophorone diisocyanate with two equivalents of HE(M)A to provide diurethane dimethacrylate monomers UDMA and IPDI-HEMA, respectively.

FIG. 3 shows real-time near-infrared spectroscopic analysis of the photopolymerization reaction kinetics and adjustable reactivity during ambient photocure condition achieved with the novel tetra-urethane di(meth)acrylate [TUD(M)A]monomers of the disclosure when formulated with either acrylic acid or methacrylic acid. Percent conversion as assessed by near-IR spectroscopy is plotted against time (seconds). The limiting conversion here applies to the ambient photocure condition and not the final conversion achieved following post curing. All the resins have a 2:1 acid to urethane functional group ratio, which each have low viscosities near ˜30 mPa·s, as shown in Table 1. The use of acidic monomer AA with a methacrylate functionalized urethane monomer very effectively raised the reactivity in the mixed acrylate/methacrylate resin compared with the analogous all-methacrylate resin.

FIG. 4 shows chemical structure of a polycaprolactone (PCL) urethane methacrylate monomer prepared from an extended PCL-diol where (m+n)˜16. This monomer was found to be compatible with acrylic acid as a comonomer where a significant excess of the acid to urethane functional group ratio was able to produce very tough and strong copolymers unlike as previously reported (Tanaka et al. 2001).

FIG. 5 shows five schemes of urethane-urethane hydrogen bonding interactions. As shown in scheme (A), extended urethane-urethane N—H—O═C hydrogen bonding interactions that requires the N—R′ bond to be in a cis configuration with the carbonyl, which is thermodynamically less favorable than the trans-urethane configuration as shown in scheme (B). The trans-urethane configuration promotes dimer urethane hydrogen bonding as shown in scheme (B) to simultaneously fully occupy the hydrogen bond donor/acceptor interactions of urethane groups. As shown in scheme (C), with a di-urethane monomer, U represents urethane linkages and * constitutes terminal reactive groups (i.e. (meth)acrylate groups). With the preferred dimer mode hydrogen bonding, extended interactions (C) are amplified by intermolecular engagement between urethane groups, which leads to high viscosity in di/multi-urethane monomers and oligomers. However, a mono-urethane monomer acts as an end-group hydrogen bond that proportionally limits the extent of intermolecular hydrogen bonding in combinations of di/multi-urethanes with mono-urethanes as shown in scheme (D). The mono-urethane monomers are inherently lower in viscosity in their bulk state since the hydrogen bonding interactions do not extend beyond dimers as shown in scheme (E).

FIG. 6 shows chemical structures of representative mono-urethane mono-(meth)acrylate monomers. For the IEM-BzOH monomer, n=1 whereas for IEMEG-BzOH, n=2.

FIG. 7A shows chemical structures of starting materials used to prepare mono-urethane di(meth)acrylate monomers, as well as structure of the common urethane dimethacrylate monomer (UDMA).

FIG. 7B shows the structures of exemplary diisocyanates that may be used for reaction with, for example, HE(M)A, or similar hydroxy (meth)acrylates such as 2-hydroxy-3-phenoxypropyl(meth)acrylate (HPPMA), hydroxypropyl(meth)acrylate (HPMA), or hydroxybutyl(meth)acrylate (HBMA), for synthesis of diurethane di(meth)acrylates according to the disclosure.

FIG. 8 shows chemical structures of linear and branched monourethane di(meth)acrylates according to Formula (IIIa).

FIG. 9 shows synthetic route and chemical structures of branched monourethane di(meth)acrylate monomers according to Formula (IIIb) with the urethane functionality located on the side-chain.

FIG. 10 shows a graph of molecular weight vs. fraction of molecules with a certain molecular weight for a polydisperse polymer sample, illustrating weight-average molecular weight (Mw), as the average molecular weight of a polydisperse polymer sample, averaged to give higher statistical weight to larger molecules; and number-average molecular weight (Mn), as the average molecular weight of a polydisperse polymer sample, averaged to give equal statistical weight to each molecule.

FIG. 11 shows chemical structures of exemplary acidic monomers including methacrylic acid (MAA), acrylic acid (AA), itaconic acid, mono-2-(methacryloyloxy)ethyl maleate, pyromellitic dianhydride glycerol dimethacrylate, 2-carboxyethyl acrylate, 2-carboxyethyl acrylate oligomer, mono-2-(methacryloyloxy)ethyl succinate, glycerol dimethacrylate/succinate adduct, 1,3-glycerol dimethacrylate/maleate adduct, bis[2-(methacryloyloxy)ethyl]phosphate, and ethylene glycol methacrylate phosphate.

FIG. 12 shows chemical structures of exemplary optional hydrophobic comonomers MMA, ISMA, cyclohexyl methacrylate, stearyl methacrylate, lauryl methacrylate, 2-ethylhexyl methacrylate, isodecyl methacrylate, isobornyl methacrylate EBDMA, and dimer acid di(meth)acrylate derivatives DA-I, DA-II, DA-III, and DA-IV.

FIG. 13 shows structures of tetraurethane monomers and anhydride comonomers (4-AETA, 4-META, 4-AMETA) used for preparation of polymer examples of the disclosure.

FIG. 14 shows unfilled and composite polymer compositions prepared from various combinations of tetraurethane monomers, acidic monomers, and anhydride comonomers, and physical properties including Modulus (GPa), Flexural strength (MPa), and Toughness (MPa) under dry and wet conditions. Conversion (%) is also shown. Surprisingly inclusion of the anhydride monomers resulted in increased Flexural Strength and Modulus under wet conditions.

FIG. 15A shows a bar graph of flexural strength (MPa) unfilled and filled composite polymer compositions comprising both acidic and anhydride comonomers along with the tetraurethane monomers [TUDA(IPDA)+3 acid (4-META and AA)]under wet and dry conditions. As shown in Table 6 (FIG. 14) the composition included an isophorone tetraurethane core, an acid: urethane ratio of 3:1, anhydride: urethane ratio of 3:2, and was either unfilled or included 60 wt % of the filler. The unfilled composition exhibited loss of flexural strength when wet (b) compared to dry conditions (a). In contrast, the filled composite composition exhibited increased flexural strength when wet (d) compared to under dry conditions (c).

FIG. 15B shows a bar graph of modulus (GPa) for unfilled and filled composite polymer compositions prepared from compositions comprising both acidic and anhydride comonomers along with the tetraurethane monomers [TUDA (IPDA)+3 acid (4-META and AA)]under wet and dry conditions. The unfilled composition exhibited some loss of modulus when wet (b) compared to dry conditions (a). In contrast, the filled composite composition exhibited substantially increased modulus when wet (d) compared to under dry conditions (c).

FIG. 16A shows a bar graph of flexural strength (MPa) unfilled and filled composite polymers prepared from compositions comprising both acidic and anhydride comonomers along with the tetraurethane monomers [XTUDA+AA+4-META (3X acid)]under wet and dry conditions. As shown in Table 6 (FIG. 14) the composition included an xylylene tetraurethane core, a 3:1 acid: urethane ratio, a 3:2 anhydride: urethane ratio, and was either unfilled or included 60 wt % of the filler. The unfilled composition exhibited loss of flexural strength when wet (b) compared to dry conditions (a). In contrast, the filled composite composition exhibited increased flexural strength when wet (d) compared to under dry conditions (c).

FIG. 16B shows a bar graph of modulus (GPa) for unfilled and filled composite polymers prepared from compositions comprising both acidic and anhydride comonomers along with the tetraurethane monomers [XTUDA+AA+4-META (3X acid)]under wet and dry conditions. The unfilled composition exhibited some loss of modulus when wet (b) compared to dry conditions (a). In contrast, the filled composite composition exhibited substantially increased modulus when wet (d) compared to under dry conditions (c).

FIG. 17A shows a bar graph of flexural strength (MPa) of unfilled polymer composition prepared from compositions comprising a tetraurethane monomer and an anhydride comonomer without acid (TUDA+4-META) having an aliphatic trimethyl hexane tetraurethane core, and an anhydride: urethane ratio of 1:2. The unfilled polymer composition exhibited increased flexural strength when wet (b) compared to dry conditions (a).

FIG. 17B shows a bar graph of modulus (GPa) of unfilled polymer composition prepared from compositions comprising a tetraurethane monomer and an anhydride comonomer without acid (TUDA+4-META) having an aliphatic trimethyl hexane tetraurethane core, and an anhydride: urethane ratio of 1:2. The unfilled polymer composition exhibited increased modulus when wet (b) compared to dry conditions (a).

### DETAILED DESCRIPTION OF THE INVENTION

The disclosure provides low viscosity polymerizable compositions comprising a urethane (meth)acrylate monomer and/or a urethane acrylate monomer, and an acidic copolymerizable monomer that may be used to prepare polymers exhibiting high strength and high toughness suitable for use in dental appliances.

As demonstrated in Sadowsky and Stansbury US20150257985A1 and U.S. 63/105,068, the addition of (meth)acrylic acid, or other suitable diluent comonomer that can coordinate with the urethane functionality, can be used to reduce the overall viscosity in proportion to its concentration. Typically, the acidic comonomer is used in stoichiometric balance with the urethane group concentration in order to achieve optimized synergistic mechanical properties in the polymers.

With respect to urethane-acid resins where functional group balance is an important factor in polymer performance, use of a higher amount of diluent monomer beyond the stoichiometric balance may diminish the strength, modulus or toughness in the expected additive fashion. Tanaka et al, Dent Mater J, 2001, 20:206-215. In particular, non-urethane reactive diluent monomers may tend to diminish the polymer toughness. Adding a non-urethane comonomer dilutes the urethane content of the resin, which typically reduces both the polymeric strength and toughness.

One surprising aspect of the present disclosure is that the polymerizable compositions comprising a multiurethane di(meth)acrylate monomer and an acidic monomer in a ratio of greater than 1:1, or 2:1 up to 10:1, or higher of the acidic moieties to urethane moieties exhibit significantly lower viscosities at ambient temperature, but also increased flexural strength, flexural modulus, and toughness post-polymerization when compared to a 1:1 stoichiometric acidic moiety to urethane moiety functional group ratio.

The disclosure provides novel tetra-urethane di(meth)acrylate monomers (TUD(M)A). Compositions comprising the TUD(M)A monomers and an acidic monomer, in an acid to urethane functional group ratio of >1:1, or about 2:1 up to 10:1 or higher, provide polymerizable compositions exhibiting low viscosity suitable for use in three-dimensional (3D) printing or other additive manufacturing technology, and surprisingly exhibit increased toughness and increased strength compared to the polymers of conventional diurethane (meth)acrylate monomers.

Specific applications include advanced 3D printing focused on dental applications through the development of extremely high performance, photocurable resin formulations. The compositions and methods provided in the present disclosure allow high performance dental applications including, but not limited to, provisional crowns, permanent crowns, bridges, denture base and teeth, including monolithic and esthetic permanent denture appliances, and directly printed orthodontic aligners, mouth guards, bite splints, whitening trays, inlays, onlays, veneers, etc.

The present disclosure provides dramatically lower viscosity urethane-based monomeric formulations that show a desirable combination of synergistically improved polymeric strength and toughness.

### Definitions

A “polymer” is a macromolecule of high relative molecular mass, the structure of which comprises the multiple repetition of units derived from molecules of low relative molecular mass.

A “branched polymer” is a type of polymer that includes side chains of repeat units connecting onto the main chain of repeat units (different from side chains already present in the monomers). A branched polymer refers to a non-linear polymer structure, but typically, not a network structure. Therefore, a trace forward from the branch point would not bridge back to the original main chain; i.e, minimal to no backbone crosslinking is present. A branched polymer would generally be soluble in an appropriate solvent.

A “crosslinked polymer” is a type of polymer that includes interconnections between chains, either formed during polymerization (by choice of monomer) or after polymerization (by addition of a specific reagent or an appropriate energy input). For example, a linear polymer can be heated or irradiated with light, electron beam, etc. to promote interchain connections. In a crosslinked polymer network, with the crosslinks serving as branch points, it is possible to trace a continuous loop back to the backbone. A crosslinked polymer network is generally a single macromolecule. The crosslinked network would be insoluble in all solvents.

A “network polymer” is a type of crosslinked polymer that includes two or more connections, on average, between chains such that the entire sample is, or could be, a single molecule. Limited crosslink connections per chain would be considered lightly crosslinked while numerous crosslinks would be considered highly (or heavily) crosslinked.

A “copolymer” is a material created by polymerizing a mixture of two, or more, comonomers. The resultant polymer molecules contain the monomers in a proportion which is related both to the mole fraction of the monomers in the starting mixture and to the reaction mechanism.

A “multi-urethane (meth)acrylate monomer” is a monomer unit comprising a multiplicity of urethane functionalities, for example, two or more, three or more, four or more, five or more, or six or more urethane functionalities, and two or more, three or more, or four or more (meth)acrylate functionalities. The multi-urethane (meth)acrylate monomer can be made by any appropriate method, including, for example, the methods according to the present disclosure, or by functionalization of multi-arm structures where there is no oligomeric mode of urethane construction involved. Multi-urethane (meth)acrylate monomers include repeated urethane building block structures as well as alternative means of introducing urethanes that do not involve oligomerization. A “chain transfer agent” is an intentionally added compound that terminates the growth of one polymer chain and then reinitiates polymerization to create a new chain. A chain transfer agent may be used as a way to limit chain length as well as the degree of crosslinking.

A “gelation time” is the time to reach the gel point (the point at which a continuous crosslinked network initially develops) during a polymerization. The gelation time can be adjustable by lowering or raising the rate of polymerization.

The “gel point” refers to the degree of conversion at which macrogelation occurs.

A “filler” is a solid extender which may be added to a polymer to modify mechanical, optical, rheological, electrical, thermal, flammable properties, or simply to act as an extender. The filler can be reactive or inert in the polymerization.

An “extender” is a substance added to a polymer to increase its volume without substantially altering the desirable properties of the polymer.

The term “ambient temperature” refers to 20-25° C., “room temperature” is 23° C., and “normal temperature” is 20° C.

The acronym “PCL” refers to polycaprolactone formed from ring-opening of a caprolactone. The term “PCL urethane (meth)acrylate monomer” refers to a monomer comprising one or more, two or more, three or more, or four or more PCL (polycaprolactone) groups (e.g., caproic acid ester; caproate, 6-(hexanoyloxy) hexanoate, polycaproate); one or more, two or more, three or more, or four or more urethane groups; and one or more, two or more, three or more, or four or more acrylate/methacylate groups. In some embodiments, the number of reactive groups in the PCL (meth)acrylate monomers per monomer molecule can be 1, 2, 3, 4 or more. PCL is a waxy solid at lower molecular weight (few thousand g/mol) and a hard solid above about 20 kg/mol. Many if not most PCL-based monomers use long enough runs of the oligomerized PCL to be semicrystalline. In the present work, those longer chain PCL segments built into (meth)acrylate-terminated monomers provide crosslinked polymers that are semicrystalline. With added MAA, the polymers remain semicrystalline and while they are quite flexible, they're also extremely low modulus and low strength. The present success using PCL urethane monomers with MAA (or similar) is limited to using just a few repeats (1-5) of the PCL spacer and both the monomers and the polymers are completely amorphous. Anything that had a longer PCL segment gave very low mechanical properties, but with short PCL segment lengths, the strength, modulus, flexibility, and toughness are generally excellent when copolymerized with an acidic monomer such as MAA.

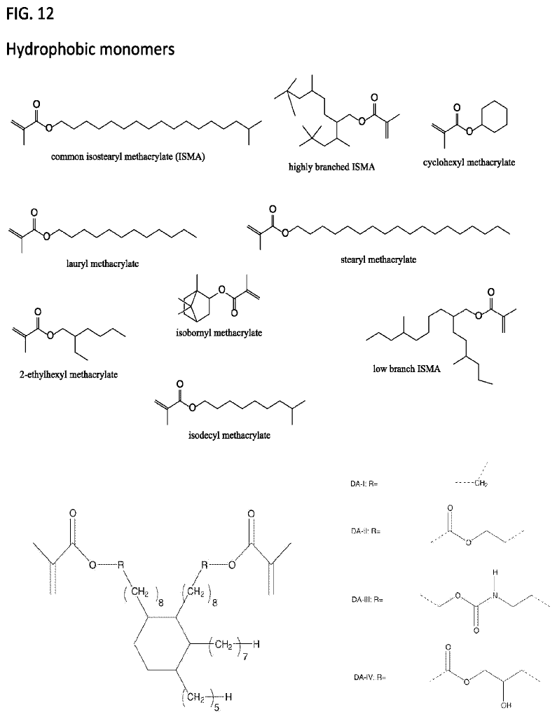

The term “hydrophobic monomer” refers to a monomer having one or more acrylate/methacrylate groups that may or may not include urethane group(s) but likely excludes other hydrophilic functional groups such as carboxylic acid, hydroxyl or other functional groups. Hydrophobicity of monomers can also be assessed and compared using the n-octanol-water distribution coefficient (log P_{o/w}). For example, methyl methacrylate has a log octanol/water partition coefficient (log K_{o/w}) of 0.79. U.S. Environmental Protection Agency. Health and Environmental Effects Profile for Methyl Methacrylate. EPA/600/x-85/364. Environmental Criteria and Assessment Office, Office of Health and Environmental Assessment, Office of Research and Development, Cincinnati, OH. 1985. In some embodiments, the hydrophobic monomer may be selected from the group consisting of isostearyl(meth)acrylate (ISMA), ethoxylated bisphenol A di(meth)acrylate (BisEMA; EBDMA), stearyl(meth)acrylate, lauryl(meth)acrylate, isodecyl(meth)acrylate, 2-ethylhexyl(meth)acrylate, isobornyl(meth)acrylate, cyclohexyl(meth)acrylate, dimer acid di(meth)acrylate, and dimer acid urethane di(meth)acrylate. In some embodiments, a highly branched version of ISMA is employed having two or more, three or more, or four or more branch points, for example, as shown in FIG. 12. Also included are hydrophobic monomers DA-I, DA-II, DA-III, and DA-IV as shown in FIG. 12, which may be produced by the method of Lemon et al, J Polymer Sci: Part A: Polymer Chemistry 2006; 44:3921-3929, which is incorporated herein by reference in its entirety.

A “low viscosity” resin refers to a resin having a viscosity at room temperature of no more than 1,000 mPa·s, no more than 500 mPa·s, no more than 300 mPa·s, or preferably no more than 100 mPa·s. In some embodiments, the low viscosity resin refers to a resin having a viscosity at room temperature of no more than no more than 500 mPa·s In some embodiments, the low viscosity resin refers to a resin having a viscosity at room temperature of no more than 300 mPa·s In some embodiments, the low viscosity resin refers to a resin having a viscosity at room temperature of no more than 100 mPa·s The threshold may be used as the basis of low viscosity although it should be noted that a resin viscosity of approximately 10 mPa·s or less may generally be desirable for ambient inkjet printing processes. In some embodiments, the low viscosity resin has a viscosity at room temperature of no more than about 50 mPa·s, no more than about 25 mPa·s, no more than 15 mPa·s, or no more than about 10 mPa·s. Low viscosity resins can speed up the printing rate on vat-based 3D printing processes as well. Typically, lower resin viscosity results in polymers that are more brittle. The compositions and methods of the present disclosure avoids that trade-off.

The term “acidic monomer” refers to a monomer having at least one acrylate/methacylate group and at least one carboxylic acid group or phosphoric acid group. The term encompasses, but is not limited to methacrylic acid (MAA), acrylic acid, itaconic acid, mono-2-(methacryloyloxy)ethyl maleate, pyromellitic dianhydride glycerol dimethacrylate, 2-carboxyethyl acrylate, 2-carboxyethyl acrylate oligomer, mono-2-(methacryloyloxy)ethyl succinate, glycerol dimethacrylate/succinate adduct, 1,3-glycerol dimethacrylate/maleate adduct, bis[2-(methacryloyloxy)ethyl]phosphate, or ethylene glycol methacrylate phosphate. The acidic monomer may be methacrylic acid. The acidic monomer may be acrylic acid.

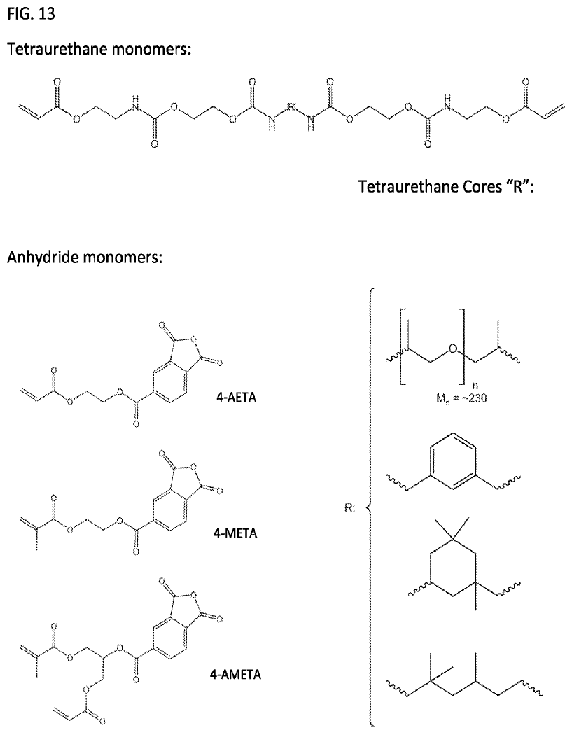

The “anhydride comonomer” refers to a monomer having at least one acrylate/methacylate group and at least one anhydride functionality. Non-limiting anhydride comonomers are shown in FIG. 13. For example, the anhydride comonomer may be 2-(acryloyloxy)ethyl 1,3-dioxo-1,3-dihydroisobenzofuran-5-carboxylate (4-AETA, 4-acryloxyethyl trimellitic anhydride), 2-(methacryloyloxy)ethyl 1,3-dioxo-1,3-dihydroisobenzofuran-5-carboxylate (4-META, 4-methacryloxyethyl trimellitic anhydride), or 1-(methacryloyloxy)-5-oxohept-6-en-2-yl 1,3-dioxo-1,3-dihydroisobenzofuran-5-carboxylate (4-AMETA).

The term “aliphatic” or “aliphatic group” as used herein means a straight-chain or branched C₁₋₂₀ hydrocarbon chain that is completely saturated or that contains one or more units of unsaturation, or a monocyclic C₃₋₈ hydrocarbon or bicyclic C₈₋₁₂ hydrocarbon that is completely saturated or that contains one or more units of unsaturation, but which is not aromatic (also referred to herein as “carbocycle” or “cycloalkyl”), that has a single point of attachment to the rest of the molecule wherein any individual ring in said bicyclic ring system has 3-7 members. For example, suitable aliphatic groups include, but are not limited to, linear or branched C₁₋₂₀ alkyl, alkenyl, alkynyl groups and hybrids thereof such as (cycloalkyl)alkyl, (cycloalkenyl)alkyl or (cycloalkyl)alkenyl. For example, aliphatic groups may be C₁₋₂₀, C₂₋₁₂, or C₄₋₈ straight or branched chain alkyl. Specific alkyl groups may include ethyl, propyl, butyl, pentyl, hexyl, heptyl, nonyl, decyl, undecyl, or dodecyl straight or branched chain groups. Exemplary aliphatic group radicals may include ethyl, n-propyl, 2,2,4-trimethyl n-hexyl, 2,4,4-trimethyl-n-hexyl, 2-methyl-n-pentyl, and 1,1,3,3-tetramethlcyclohexyl.

The terms “alkoxy,” “hydroxyalkyl,” “alkoxyalkyl” and “alkoxycarbonyl,” used alone or as part of a larger moiety include both straight and branched chains containing one to twelve carbon atoms. The alkoxyalkyl may be, for example, a polyethylene ether or polypropylene ether. Exemplary alkoxyalkyl may include 1,2-diethoxyethane, ethoxyethane, 1-ethoxy-2-(2-ethoxyethoxy) ethane, and 1-propoxypropane.

The terms “alkenyl” and “alkynyl” used alone or as part of a larger moiety shall include both straight and branched chains containing two to twelve carbon atoms, or two to eight carbon atoms having at least one double bond or triple bond, respectively.

The term “heteroatom” means nitrogen, oxygen, or sulfur and includes any oxidized form of nitrogen and sulfur, and the quaternized form of any basic nitrogen.

The term “aryl” used alone or in combination with other terms, refers to monocyclic, bicyclic or tricyclic carbocyclic ring systems having a total of five to fourteen ring members, wherein at least one ring in the system is aromatic and wherein each ring in the system contains 3 to 8 ring members. The term “aryl” may be used interchangeably with the term “aryl ring”. The term “aralkyl” refers to an alkyl group substituted by an aryl. The term “aralkoxy” refers to an alkoxy group substituted by an aryl. The aryl ring may be a substituted or unsubstituted phenyl radical. The aryl ring may be a xylyl radical, such as a 1,3-xylenyl radical or a 1,4-xylenyl radical.

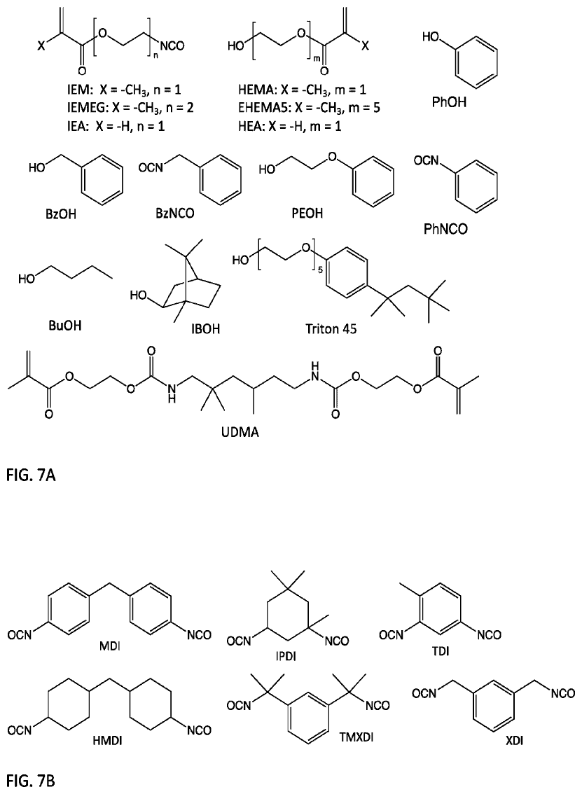

The term “(meth)acrylate” when used in a chemical name is intended to encompass both methacrylate and acrylate chemical structures in both the monomeric and polymeric states. For example, “HE(M)A” is meant to encompass both HEMA (2-hydroxyethyl methacrylate; X=—CH₃, m=1), and HEA (2-hydroxyethyl acrylate; X=H, m=1), as shown in FIG. 7A. “HPP (M) A” is meant to encompass both 2-hydroxy-3-phenylpropyl methacrylate and 2-hydroxy-3-phenylpropyl acrylate. “HP (M) A” is meant to encompass both 2-hydroxypropyl methacrylate and 2-hydroxypropyl acrylate. “HB(M)A” is meant to encompass both 2-hydroxybutyl methacrylate and 2-hydroxybutyl acrylate.

Some synthetic polymers may have a distribution of molecular weights (MW, grams/mole). Polydispersity describes a polymer consisting of molecules with a variety of chain lengths and molecular weights. The width of a polymer's molecular weight distribution is estimated by calculating its polydispersity, Mw/Mn. The closer this approaches a value of 1, the narrower is the polymer's molecular weight distribution.

The “weight-average molecular weight” (Mw) is the average molecular weight of a polydisperse polymer sample, averaged to give higher statistical weight to larger molecules; calculated as Mw=SUM (Mi² Ni)/SUM (Mi Ni), where Ni is the number of molecules of molecular weight Mi. One technique used to measure molecular weights of polymers is light scattering. A light shining through a very dilute solution of a polymer is scattered by the polymer molecules. The scattering intensity at any given angle is a function of the second power of the molecular weight. Consequently, because of this “square” function, large molecules will contribute much more to the molecular weight that we calculate than small molecules.

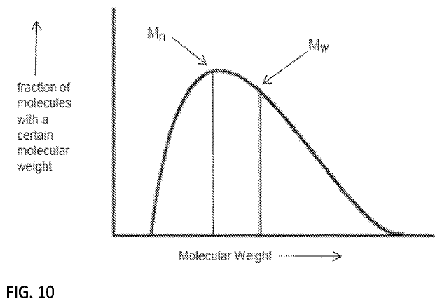

The “number-average molecular weight” (Mn) is the average molecular weight of a polydisperse polymer sample, averaged to give equal statistical weight to each molecule; calculated as Mn=SUM (Mi Ni)/SUM (Ni), where Ni is the number of molecules of molecular weight Mi. Relationship of Mn and Mw is shown in FIG. 10.

One problem to be solved was to develop new polymerizable compositions having low viscosities amenable to 3D printing, and exhibit improved flexural strength and toughness post-polymerization, for example, when compared to traditional urethane (meth)acrylate compositions comprising one or two urethane groups, for example, UDMA or IPDI-HEMA, shown in FIG. 2.

### Monomers

The disclosure provides low viscosity polymerizable compositions comprising a urethane (meth)acrylate monomer and/or a urethane acrylate monomer, and an acidic monomer.

The urethane (meth)acrylate monomer may be a mono-, di-, tri-, tetra- or other multi-urethane (meth)acrylate monomer. The urethane (meth)acrylate monomer may be a mono-, di-, tri-, tetra- or other multi-urethane multi (meth)acrylate monomer. For example, the urethane (meth)acrylate monomer may be a mono-, di-, tri-, tetra- or other multi-urethane di(meth)acrylate monomer.

The urethane acrylate monomer may be a mono-, di-, tri-, tetra- or other multi-urethane acrylate monomer. The urethane acrylate monomer may be a mono-, di-, tri-, tetra- or other multi-urethane multi-acrylate monomer. For example, the urethane acrylate monomer may be a mono-, di-, tri-, tetra- or other multi-urethane diacrylate monomer.

For example, the low viscosity polymerizable compositions may comprise a mono-, di-, tri-, tetra- or other multi-urethane di(meth)acrylate monomer and an acidic monomer.

In some examples, the low viscosity polymerizable compositions may comprise a mono-, di-, tri-, tetra- or other multi-urethane di(meth)acrylate monomer and a mono-urethane mono(meth)acrylate monomer without an acidic monomer. Strength and toughness could be increased, for example, by adding an acidic monomer. In some embodiments, the low viscosity polymerizable composition comprises a mono-, di-, tri-, tetra- or other multi-urethane di(meth)acrylate monomer, a mono-urethane mono(meth)acrylate monomer, and an acidic monomer.

### Tetraurethane (Meth)Acrylate Monomers and Reactive Oligomers

The disclosure provides novel tetraurethane (meth)acrylate (TUD(M)A) monomers. Compositions comprising the TUD(M)A monomers and acidic monomers have been found to exhibit low viscosity suitable for 3D printing, and exhibit desirable flexural strength, flexural modulus, and improved toughness when the ratio of acidic to urethane functional groups is, for example, >1:1, in particular when the ratio is 2:1, or higher.

In some embodiments, TUD(M)A monomers according to Formula (I) are provided

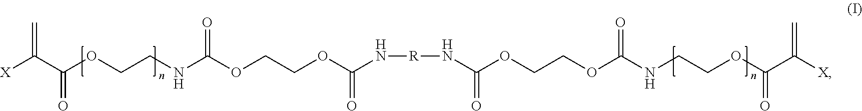

wherein X is —H or —CH₃; n is 1, 2, 3 or 4; and R is aliphatic, alkoxyalkyl, or alkylarylalkyl core group. In some embodiments, a TUDMA monomer according to Formula (I) is provided wherein wherein X is —H or —CH₃; n is 1 or 2; and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group. R may be ethyl, n-propyl, 2,2,4-trimethyl n-hexyl, 2,4,4-trimethyl-n-hexyl, 2-methyl-n-pentyl, or 1,1,3,3-tetramethlcyclohexyl radical core group.

In some embodiments, X is —H or —CH₃; n is 1 or 2, and R is a core radical selected from the group consisting of

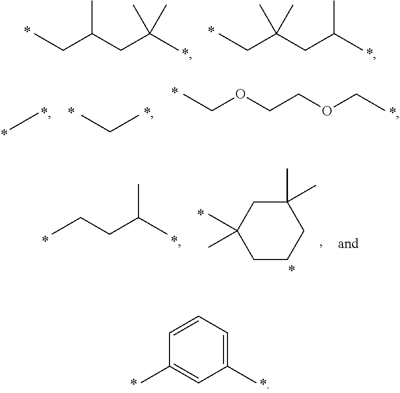

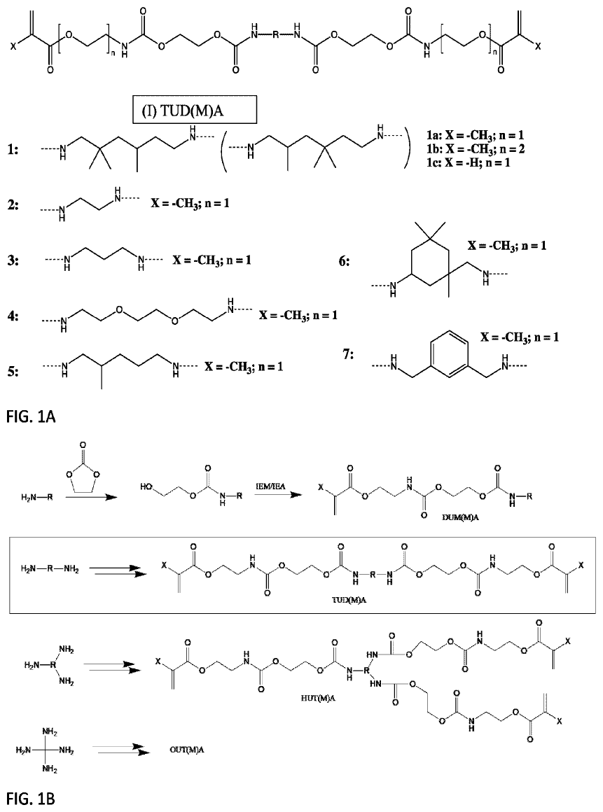

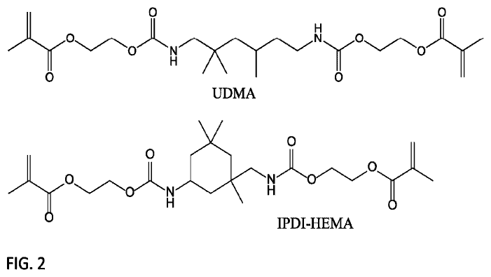

New tetra-urethane di(meth)acrylate monomers (e.g., TUDMA1a and TUDMA1b; FIG. 1) are provided herein that can be prepared by the two-step reaction of an aliphatic diamine or aryl diamine, for example, trimethylhexyldiamine, ethyldiamine, propyldiamine, methylpentyldiamine, and the like (see, e.g., FIG. 1A.1 to 7), with two equivalents of ethylene carbonate to make the internal urethane linkages, followed by addition of either isocyanatoethyl(meth)acrylate (IEM) or isocyanatoethoxyethyl(meth)acrylate (IEMEG). Structures of IEM and IEMEG are shown in FIG. 7A. Exemplary structures of tetra-urethane di(meth)acrylate monomers are also shown in FIG. 13.

The disclosure also provides other multi-urethane (meth)acrylate monomers comprising close symmetric placement of two urethane groups per segment that is integrated with a polymerizable group. This type of structure for use in urethane-based (meth)acrylate monomers may facilitate a very positive interaction with the acidic comonomer. While the poly (TUDMA) homopolymers were not found to be particularly strong or tough without the acidic comonomer, copolymerization with MAA or AA produced polymers with excellent mechanical properties. The disclosure provides similar multi-urethane (meth)acrylate monomers compared to the tetra-urethane (meth)acrylate of Formula (I) comprising di-urethane, tetra-urethane (as shown), hexa-urethane, etc., by reacting an amine core where the starting amine has mono-, di-, tri-, etc. amino functionality. Multi-urethane monomers can be made as in the tetra-urethane approach or by functionalization of multi-arm structures where there is no oligomeric mode of urethane construction involved. The term “multi-urethane” includes compounds comprising a multiplicity of or repeated urethane building block structures as well as alternative means of introducing urethanes that does not involve oligomerization.

### Di-Urethane Monomers

The disclosure provides low viscosity polymerizable compositions comprising diurethane (meth)acrylate monomers. The diurethane di(meth)acrylate may be a commercially available diurethane di(meth)acrylate or may be obtained synthetically. However, viscosity reduction into the range defined here as low viscosity is dependent on the addition of an acidic comonomer as a reactive diluent.

In some embodiments, the di-urethane di(meth)acrylate may be obtained commercially, for example, UDMA or IPDI-HEMA. In some embodiments, the diurethane di(meth)acrylate may be synthesized, for example, HE(M)A could be reacted with diisocyanates as shown in FIG. 7B to make the diurethane di(meth)acrylates HE(M)A-MDI, HE(M)A-IPDI, HE(M)A-TDI, HE(M)A-HMDI, HE(M)A-TMXDI, or HE(M)A-XDI, respectively.

The diurethane di(meth)acrylate monomer may be a polycaprolactone (PCL)diurethane (meth)acrylate. PCL diurethane di(meth)acrylates are disclosed in US provisional application U.S. 63/105,068. An exemplary PCL diurethane (meth)acrylate is shown in FIG. 4. This PCL diurethane (meth)acrylate monomer, in which m+n˜ 16, was prepared from a polycaprolactone (PCL)-diol with molecular weight of approximately 2000 g/mol that is first reacted with two-equivalents of IEM to produce the di-urethane dimethacrylate with two extended oligomeric PCL segments, as described in U.S. 63/105,068. Previously this PCL diurethane (meth)acrylate was only used with MAA. A composition comprising the PCL monomer of FIG. 4 and AA was found to produce a significantly lower viscosity comonomer mixture. When this PCL monomer is formulated with a 10-fold functional group excess of AA acid to urethane functional groups, a very low viscosity (8.58±0.38 mPa·s) resin is obtained.

### Mono-Urethane Monomers

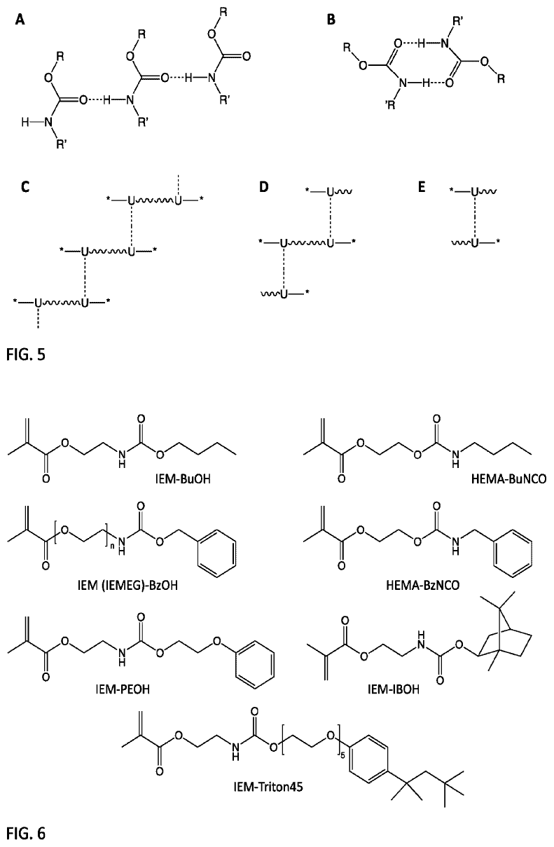

In molecular structures that contain urethane linkages, the presence of free, non-hydrogen bonded urethane groups are minimal. Lemon MT, Jones MS, Stansbury J W. Hydrogen bonding interactions in methacrylate monomers and polymers. Journal of Biomedical Materials Research Part A 2007; 83A (3): 734-46. The hydrogen bonding interactions between urethane groups can involve urethane-urethane dimer formation, or the both enthalpically and entropically less favored stacking of multiple urethane groups, as shown in FIG. 5.

In the case of di-urethane compounds, there is some degree of intramolecular urethane-urethane hydrogen bonding, but it is the intermolecular hydrogen bonding between urethane groups that promotes extended interactions that promotes high monomer viscosity.

With mono-urethane monomers, the rapid, transient formation of urethane-urethane dimer hydrogen bonds only doubles the apparent molecular mass of a relatively low molecular weight compound, which has only a modest effect on monomer viscosity as compared with the extended intermolecular hydrogen bonding associated with di- and multi-urethane materials (FIG. 1A and 2) that make these compounds behave as if they were significantly higher molecular weight structures.

There are limited examples of mono-urethane monomers in the literature record and in these available examples, the primary rationale is the examination of the unusually rapid reaction kinetics associated with these monomers as homopolymers and copolymers.³⁻¹³ In those prior studies, there is no attention paid to monomer viscosity in part because several of the monomers studied are crystalline solids. There is also no consideration given to mechanical property potential of these monomers as homopolymers and little thought of how these mono-urethane mono(meth)acrylates function in urethane-based resin systems. Specifically, there is no indication of the use of these monomers in copolymerizations to tailor polymer modulus and strength and certainly no thought of using these in high performance urethane (meth)acrylate resins with extreme toughness.

In the present disclosure, mono-urethane mono(meth)acrylates can be used as a means to reduce viscosity of di-/multi/oligomeric urethane (meth)acrylates while retaining high urethane content and controllably reducing the covalent crosslink density that can favorably increase flexibility and toughness. The additional use of an acidic comonomer as a reactive diluent capable of strong, non-covalent interactions with urethane groups to further reduce viscosity of the resin and promote a physical reinforcement of the corresponding copolymers that provides enhanced strength, modulus, and toughness.

To provide some examples, a variety of mono-urethane mono-(meth)acrylate monomers were synthesized using highly efficient alcohol-isocyanate reactions as shown in the present Example 3, although other non-isocyanate routes to these monomers are available. For example, see Meng et al, Polymer 2017; 109:146-59; Rokicki et al, Polymers for Advanced Technologies 2015; 26 (7): 707-61; and Zareanshahraki et al, Progress in Organic Coatings 2020; 138.

Structures of the synthesized mono-urethane mono(meth)acrylate monomers are provided in FIG. 6. The monomers presented here were obtained by stoichiometric reaction of isocyanatoethyl methacrylate (IEM; FIG. 7A) with a variety of alkyl and aryl alcohols, as shown in FIG. 7A. Resultant mono-urethane monomers include IEM-BuOH, HEMA-BuNCO, IEM-BzOH (n=1), IEMEG-BzOH (n=2), HEMA-BzNCO, IEM-PEOH, IEM-IBOH, and IEM-Triton45. Structural variation was readily achieved by use of the ethylene glycol extended methacrylate-functionalized isocyanate (IEMEG) as shown for the reaction product of IEMEG and benzyl alcohol (BzOH).

Alternatively, the mono-urethane monomers can be prepared from HEMA or related hydroxy methacrylates by reaction with an alkyl or aryl isocyanate. This approach demonstrates that isomeric monomer pairs that differ only in the configuration of the urethane group with respect to the polymerizable (meth)acrylate group are easily achieved as indicated by the IEM-BzOH and HEMA-BzNCO as well as IEM-BuOH and HEMA-BuNCO monomers. (FIG. 7). Replacement of the methacrylate groups with acrylate functionality is easily achieved by substitution of isocyanatoethyl acrylate (IEA) in place of IEM or by use of 2-hydroxyethyl acrylate (HEA) in place of HEMA. There are numerous further examples of mono-urethane monomers that could be constructed in this manner.

The mono-urethane mono(meth)acrylate monomers may be obtained by stoichiometric reaction of isocyanatoethyl methacrylate (IEM; FIG. 7) with a variety of alkyl and aryl alcohols. Structural variation can readily be achieved by use of the ethylene glycol extended methacrylate-functionalized isocyanate (IEMEG) as shown for the reaction product of IEMEG and benzyl alcohol (BzOH). Mono-urethane mono(meth)acrylate monomers are provided.

The mono-urethane mono(meth)acrylates may be a compound according to Formula (II).

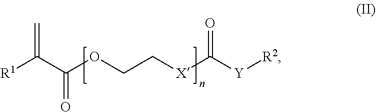

wherein n=1 or 2; R1 is —H or —CH₃; X′ is O or N; Y is N when X′=O, or Y is O when X′=N; R2 is aliphatic, benzyl, alkoxyaryl, or polyalkoxyaryl. In some embodiments, R² is selected from the group consisting of C₁-C₁₀ straight or branch chain alkyl, C₅-C₁₀ substituted or unsubstituted cycloalkyl. In some embodiments, R² is n-butyl, benzyl, isobornyl, or 1-(4-(2,4,4-trimethylpentan-2-yl) phenoxy)-3,6,9, 12-tetraoxatetradecane. The mono-urethane mono(meth)acrylates may be selected from IEM-BuOH, HEMA-BuNCO, IEM-BzOH, IEMEG-BzOH, HEMA-BzNCO, IEM-PEOH, IEM-IBOH, or IEM-Triton45, as shown in FIG. 6.

The mono-urethane mono(meth)acrylate monomers of FIG. 6 are all liquid at room temperature and their bulk viscosities are given in Table 2. Mono-urethane mono(meth)acrylate monomers exhibit dramatically lower ambient viscosity compared with a conventional di-urethane dimethacrylate (UDMA; prepared by the reaction of 2,2,4-(2,4,4)-trimethylhexyldiisocyanate with two equivalents of 2-hydroxyethyl methacrylate), which is considered to have a relatively low viscosity among urethane monomers at ˜8900 mPa·s, and certainly lower than that of reactive urethane oligomers, which are typically measured at elevated temperatures because of the excessively high viscosity values involved.

For the varied mono-urethane mono-methacrylate monomers reported here, the ambient bulk viscosities are low, but also cover a significant range at the low end of the viscosity scale. For example, whereas benzyl methacrylate, which lacks any hydrogen bond donor groups, has a viscosity of ˜3 mPa·s, the viscosity of the benzyl mono-urethane methacrylate is about an order of magnitude greater in viscosity due to both hydrogen bond donor and acceptor character associated with the urethane linkage. It is somewhat surprising that the isobornyl urethane methacrylate is approximately 50-fold more viscous than isobornyl methacrylate (˜8 mPa·s), which suggests the sterically demanding isobornyl groups interfere with simple urethane-urethane dimer formation and instead favor the more extended hydrogen bonding that promotes higher viscosity. The amphiphilic structure of the Triton45 also leads to a higher viscosity mono-urethane monomer likely due to the hydrogen bonding interaction between the urethane and the extended ether groups. The IEM-BuOH monomer may well be the lowest viscosity bulk urethane (meth)acrylate monomer possible because the analog prepared with IEM and ethyl alcohol is a crystalline solid at room temperature. Berchtold KA, Nie J, Stansbury J W, et al. Macromolecules 2004; 37 (9): 3165-79.

Compositions comprising mono-urethane mono(meth)acrylate monomers and conventional di/multi-urethane (meth)acrylate monomers or oligomers are provided. The mono-urethane mono-(meth)acrylates are useful as co-monomers with conventional di/multi-urethane (meth)acrylate monomers and oligomers as a way to control the covalent network density without sacrificing the overall urethane group density while effectively lowering resin viscosity. Specific mono-urethane mono-(meth)acrylate monomers are useful for copolymerizations as a means to target certain polymer properties such as glass transition temperature, modulus, refractive index or hydrophilicity, and the structures presented here would produce a range of these and other polymer properties. The mono-urethane mono-(meth)acrylate monomers are also useful as homopolymers, or for use in copolymerizations in place of non-urethane containing reactive diluent monovinyl monomers since the urethane-functionality introduced here has been shown to accelerate polymerization, particularly in processes involving resin photopolymerization.

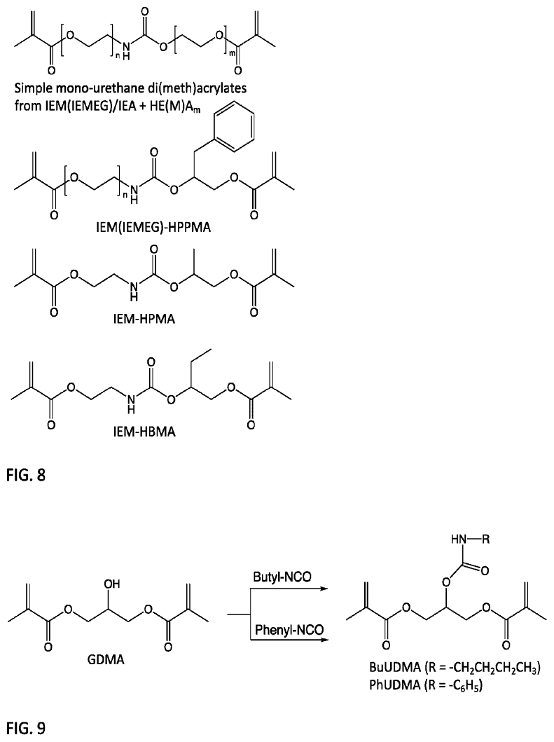

Mono-urethane di(meth)acrylate monomers are provided. The extension of the mono-urethane mono-(meth)acrylates, which produce linear polymer, to a crosslinkable version that retains the single urethane group between two (meth)acrylate functional groups was also pursued. In the present disclosure, mono-urethane di(meth)acrylates may be used to reduce viscosity while keeping both the urethane concentration and the crosslink density high. The additional use of an acidic comonomer as a reactive diluent capable of strong, non-covalent interactions with urethane groups to further reduce viscosity of the resin and promote a physical reinforcement of the corresponding copolymers that provides enhanced strength, modulus, and toughness. A series of mono-urethane di(meth)acrylates was prepared from the pairwise combination of IEM or IEA with HEMA or HEA. These four monomers as well as several additional variations that can similarly be obtained by using extended versions of either or both the isocyanato (meth)acrylate or the hydroxy (meth)acrylate components, as represented in FIG. 8. Branched crosslinkers can readily be obtained by using hydroxy (meth)acrylates such as 2-hydroxy-3-phenoxypropyl(meth)acrylate (HPPMA), hydroxypropyl(meth)acrylate (HPMA) and hydroxybutyl(meth)acrylate (HBMA) in reactions with (meth)acrylate-functional isocyanates. With regard to branched urethane monomer structures, the placement of the mono-urethane group in a side-chain off the crosslinker unit of a di(meth)acrylate was further investigated. Through use of glycerol dimethacrylate (GDMA) with either butyl isocyanate (BuNCO) or phenyl isocyanate (PhenyINCO), the side-chain urethanes were prepared as shown in FIG. 9.

The mono-urethane di(meth)acrylate may be a compound according to Formula (IIIa):

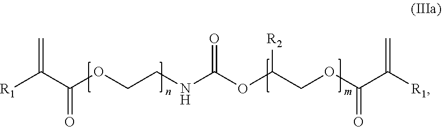

wherein R₁=—H, —CH₃; n=1 or 2; m=1-5; and R₂ is an aliphatic, aryl, alkylaryl group. In some embodiments, R₂=H, Me, Et, nPr, iPr, nBu, sBu, tBu, Phe, or Bzl. In some embodiments, R₂=Me, Et, or Bzl.

The mono-urethane di(meth)acrylate may be a branched compound according to Formula (IIIb), having the urethane functionality in a side-chain:

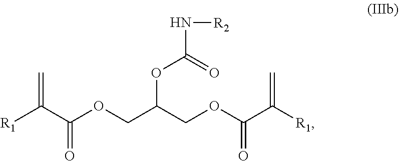

wherein R₁=—H, —CH₃; n=1 or 2; m=1-5, R₂=aliphatic, aryl, alkylaryl In some embodiments, R₂=Me, Et, nPr, iPr, nBu, sBu, tBu, Phe, or Bzl. In some embodiments, R₂=nBu or Phe.

The monomeric and polymeric properties of the exemplary series of synthesized mono-urethane di(meth)acrylates according to Formula (IIIa) is shown in Table 3. The homopolymers as well as the MAA copolymers produced as equimolar mixtures (except as otherwise noted) are described. The neat room temperature viscosities are provided along with the degree of conversion achieved with an ambient photopolymerization under the following conditions: 0.1 wt % 2,2-dimethoxy-2-phenylacetophenone (DMPA) photoinitiator; medium pressure mercury arc UV light with 365 nm filter to give an incident irradiance of 100 mW/cm². The ambient conversion values are reported after 10 minute continuous light exposure using real-time near-infrared spectroscopy to monitor the =CH₂ (meth)acrylate reactive group at ˜6165 cm⁻¹. The polymer and copolymers were then also post-cured in a commercial light-curing oven for 1 hour at 80° C. with concurrent exposure to 365 and 405 nm light sources. All the polymeric mechanical properties are reported for post-cured materials based on 3-point bend testing of 2×2×25 mm specimens (n=5-8) on a 20 mm span with a crosshead speed of 1 mm/min. The tensile properties of two of the mono-urethane dimethacrylate homopolymers and the corresponding MAA-diluted (1:1 molar ratio) are shown in Table 4.

### Acidic Comonomers

The disclosure provides polymerizable resin compositions comprising a urethane (meth)acrylate monomer and an acidic comonomer. The addition of an acidic comonomer, such as methacrylic acid (MAA), provides a means to markedly suppress the viscosity of urethane monomers. In the case of the UDMA/MAA mixture shown in Table 1, the large reduction in viscosity is a consequence of the carboxylic acid interaction with the urethane functionality, but also because two equivalents of MAA are required to coordinate with the two urethane groups in UDMA; wherein certain other examples recite equimolar mixtures. As a result of the preferential interaction between acid and urethane functionality, any extended urethane-urethane hydrogen bonding is largely disrupted. While not universal, both alkyl and aryl mono-urethane mono(meth)acrylates when combined with MAA can provide viscosities as low as non-hydrogen bonding monomers that typically yield polymers with less desirable properties.

In some di-/multi-urethane materials, higher concentrations of the acidic comonomer can provide equivalent or even enhanced mechanical properties for the copolymers compared with balanced urethane: acid functional group proportions. In these cases, the higher amount of diluent monomer beyond the stoichiometric balance does not diminish the strength, modulus or toughness in the expected additive fashion (see Tanaka et al, Dent Mater J, 2001, 20:206-215), but does provide a route to achieve high-performance urethane-based resin formulations of significantly reduced viscosity. In terms of lowering viscosity, the acidic diluent monomer is not just diluting the urethane-urethane interactions that are responsible for the high viscosity of urethane-based compositions. That dilution effect is how the conventional diluent comonomers are used in urethane materials. With acidic monomers, the acid preferentially interacts with the urethane and thus it displaces the urethane-urethane interactions, so it reduces viscosity more effectively than a conventional diluent at the same concentration.

The present disclosure also provides compositions and methods in which the acidic monomer acrylic acid (AA) can lower the viscosity of traditional urethane (meth)acrylate monomer formulations to a greater extent than the same molar concentration of acidic monomer MAA. In particular, the urethane monomer derived from isophorone diisocyanate and HEMA (IPDI-HEMA; FIG. 2) has a higher viscosity (983±69 mPa·s) when diluted with MAA at a 1:1 acid to urethane functional group ratio as compared with the equivalent balance of AA (877±19 mPa·s) added to IPDI-HEMA. In contrast, with conventional UDMA, the 1:1 acid to urethane dilution with either MAA or AA provides equivalent viscosities of 118±2.7 mPa·s or 120±11 mPa·s, respectively. It is also noteworthy that because AA is ˜15% lower in molecular weight relative to MAA, that the stoichiometric balance of acid to urethane functional groups means a significantly lower mass or volumetric proportion of the AA diluent monomer is involved compared with MAA. Thus, even equivalent viscosity between corresponding AA and MAA diluted urethane formulations provides an indication of the enhanced viscosity reduction potential of AA when used with urethane comonomers. It should also be recognized that even small amounts of viscosity reduction could be critical in both vat-based and ink-jetting 3D printing platforms.

To further highlight the differential interaction between acidic monomers AA and MAA when formulated at 1:1 acid to urethane ratio with IPDI-HEMA, the modulus and flexural strength of these copolymers are 3.66±0.67 GPa and 89.0±24.3 MPa with MAA and 4.63±0.33 GPa and 172.1±34.5 MPa for the AA-diluted resin. It is unexpected that AA would provide improved mechanical properties in copolymers compared with use of MAA. Methacrylates are generally used in place of acrylates for their contribution to higher glass transition temperature and corresponding higher strength and modulus in the polymeric state relative to acrylate polymers and copolymers. Notably, the strength and modulus difference favoring copolymerization with AA vs MAA is quite substantial, which again is unexpected.

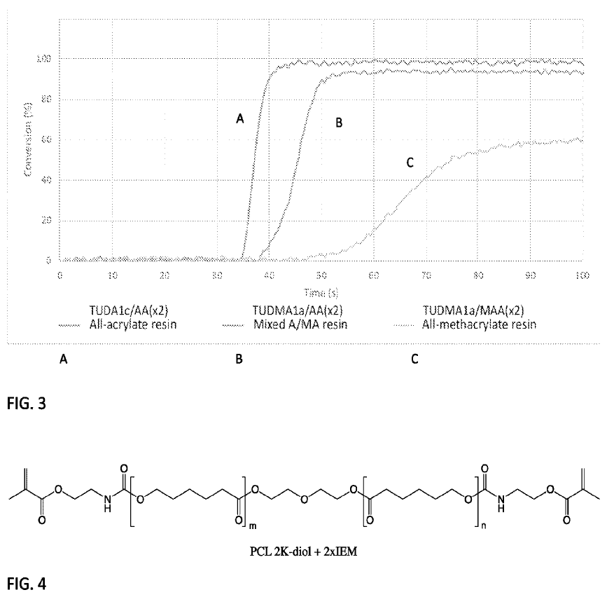

The use of acidic monomer AA with a methacrylate functionalized urethane monomer has been found to very effectively raise the reactivity in the mixed acrylate/methacrylate resin compared with the analogous all-methacrylate resin as shown in FIG. 3. FIG. 3 shows real-time near-infrared spectroscopic analysis of the photopolymerization reaction kinetics and limiting conversion achieved with the novel tetra-urethane di(meth)acrylate [TUD(M)A]monomers of the disclosure when formulated with either acrylic acid or methacrylic acid. Percent conversion as assessed by near IR spectroscopy is plotted against time (seconds). All the resins have a 2:1 acid to urethane functional group ratio, which each have low viscosities near ˜30 mPa·s, as shown in Table 1. The resin composition comprising TUD(M)A monomer according to Formula (I) 1c (having X=—H, n=1) and acidic monomer AA, with an acid to urethane functional group ratio of 2, (Composition A; all acrylate resin) exhibited about 95+% conversion that progressed during an interval less than 10 seconds. The resin composition comprising TUD(M)A monomer according to Formula (I) 1a (having X=—CH₃, n=1) and acidic monomer AA, with an acid to urethane functional group ratio of 2, (Composition B; mixed acrylate/methacrylate resin) exhibited about 90+% conversion during an interval slightly greater than 10 seconds. The resin composition comprising TUD(M)A monomer according to Formula (I) 1a (having X=—CH₃, n=1) and acidic monomer MAA, with an acid to urethane functional group ratio of 2, (Composition C; all methacrylate resin) exhibited about 60% conversion over about 40 seconds. The reactivity of the all-acrylate resin and the mixed acrylate/methacrylate resin was significantly improved compared to the all-methacrylate resin, as shown in FIG. 3.

This aspect may be exploited to significantly enhance the photocure-based printing efficiency without significant sacrifice to high-performance properties of the polymer and in some cases, the acrylate or mixed (meth)acrylate resins offer much higher mechanical properties as compared with the all-methacrylate version of these resins, which again is not expected.

Also notable was the use of acidic monomer acrylic acid in place of methacrylic acid as a comonomer raised the ambient photocure conversion limit dramatically when used with either an acrylate or a methacrylate functionalized urethane monomer. The near quantitative conversion achieved under ambient photocuring conditions with TUD(M)A used together with AA may avoid or simplify the need for post-cure processing of a 3D printed part. The potential to use low irradiance (10 mW/cm² of 365 nm UV light) with minimal photoinitiator (0.1 wt % DMPA) to produce these very high modulus polymers at 95+% conversion is yet another unexpected result here. Typically, the mobility restrictions associated with polymer network vitrification occur at much lower conversion levels unless a rubbery, low modulus polymer is produced. Here, the mobility limit and the near-complete consumption of the reactive groups both occur simultaneously for the all-acrylate system and nearly at full conversion for the mixed acrylate/methacrylate combination.

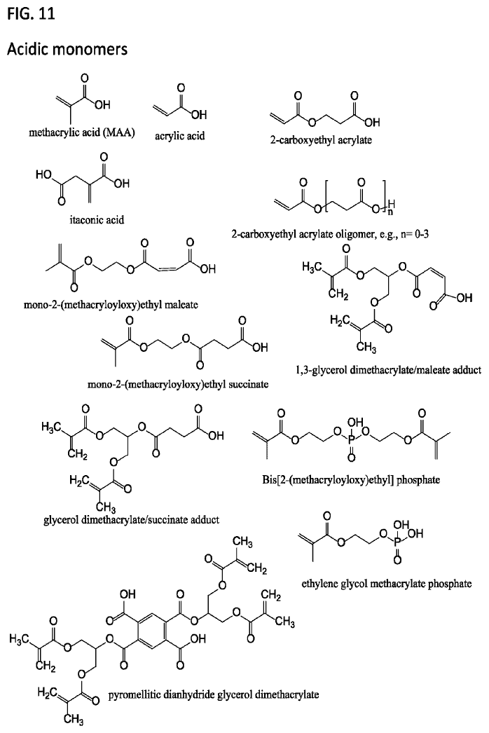

The acidic comonomer may be selected from the group consisting of acrylic acid, methacrylic acid (MAA), itaconic acid, mono-2-(methacryloyloxy)ethyl maleate, pyromellitic dianhydride glycerol dimethacrylate, 2-carboxyethyl acrylate, 2-carboxyethyl acrylate oligomer, mono-2-(methacryloyloxy)ethyl succinate, glycerol dimethacrylate/succinate adduct, 1,3-glycerol dimethacrylate/maleate adduct, bis[2-(methacryloyloxy)ethyl]phosphate, or ethylene glycol methacrylate phosphate. Structures of exemplary acidic comonomers are shown in FIG. 11.

The ratio of the acidic functionality from the acidic monomer to that of the urethane groups in the urethane (meth)acylate monomer may be in a molar ratio of from about 1:1 to about 12:1 or higher, >1:1 up to 10:1 or higher, or from about 2:1 to about 10:1, 2:1, 3:1, 4:1, 5:1, 6:1, 7:1, 8:1, 9:1, or 10:1, or any value in between.

### Optional Comonomers

The compositions of the present disclosure may optionally further include a hydrophobic comonomer, or a comonomer that raises or lowers the glass transition temperature. These types of comonomers may be optionally employed, for example, to achieve certain targeted properties. In some embodiments, the polymerizable resin composition may further comprise one or more hydrophobic monomers. For example, the hydrophobic monomer may be selected from the group consisting of isostearyl(meth)acrylate (ISMA), ethoxylated bisphenol A di(meth)acrylate (EBDMA), stearyl(meth)acrylate, lauryl(meth)acrylate, isodecyl(meth)acrylate, 2-ethylhexyl(meth)acrylate, isobornyl(meth)acrylate, cyclohexyl(meth)acrylate, dimer acid di(meth)acrylate, and dimer acid urethane di(meth)acrylate. In some embodiments the hydrophobic monomer is an ISMA. In some embodiments, the ISMA has one or more, two or more, three or more, four or more, or five or more branch points. Three versions of ISMA are illustrated in FIG. 12. All have at least one branch point along the C18 alkyl chain, which renders the monomer an amorphous liquid rather than a waxy, semi-crystalline solid in the case of stearyl(meth)acrylate. In some embodiments, the ISMA has three or more branch points. In some embodiments, the ISMA has five or more branch points. In some embodiments, the ISMA has a structure shown in FIG. 12. Common ISMA has a single branch point. The branching may be important to avoid crystallinity involving the C18 chains. However, the highly branched ISMA works very well as a significantly hydrophobic comonomer that interdigitates the multi-branch points into the overall polymer network, without sacrificing the mechanical strength potential that the other urethane and acidic monomers contribute. The weight ratio of urethane (meth)acrylate comonomers and/or urethane acrylate comonomers, plus acidic monomers compared to the optional hydrophobic comonomers may be in a range of from about 99:1 to about 80:20; 98:2 to 85:15; or 95:5 to 90:10.

The compositions of the disclosure may optionally include a mono-urethane monovinyl comonomer or mono-urethane divinyl comonomer, that can be blended with oligomeric urethane (meth)acrylates, di-urethane, or multi-urethane monomers as a means to control resin viscosity. This effect represents a practical, useful approach to modify commercial urethane photopolymers, even before adding acidic comonomers that would further reduce resin viscosity.

### Initiators

The polymerization of the monomers may be initiated by any suitable method of generating free-radicals such as by thermally induced decomposition of a thermal initiator such as an azo compound, peroxide or peroxyester. Alternatively, redox initiation or photo-initiation can be used to generate the reactive free radicals. Therefore the polymerization mixture also preferably contains a polymerization initiator which may be any of those known and conventionally used in free-radical polymerization reactions, e.g. azo initiators such as 2,2′azobis(isobutyronitrile) (AIBN), azobis(2-methylbutyronitrile), azobis(2,4-dimethylvaleronitrile), 4,4-azobis(4-cyanovaleric acid), 1,1′-azobis(cyclohexanecarbonitrile); peroxides such as benzoyl peroxide, dilauroyl peroxide, tert-butyl peroxyneodecanoate, dibenzoyl peroxide, 2,2-bis(tert-butylperoxy) butane, 1,1-bis(tert-butylperoxy) cyclohexane, 2,5-bis(tert-butylperoxy)- 2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butylperoxy isopropyl carbonate, cyclohexanone peroxide, dicumyl peroxide, lauroyl peroxide, 2,4-pentanedione peroxide, peracetic acid, cumyl peroxide, tert-butyl peroxy-2-ethyl hexanoate, tert-butyl peroxy diethyl acetate, tert-amyl peroxybenzoate, and tert-butyl peroxy benzoate. In some embodiments, the thermal initiator is benzoyl peroxide (BPO). BPO has been effectively used at concentrations between 0.5 and 2 wt % relative to the resin phase. The preferred concentration is 1.35-1.85 wt %. In some embodiments, the thermal initiator is AIBN.

In another aspect, the initiator is a redox (reduction-oxidation) pair of initiators. Redox initiator systems use both a primary initiator and a chemical reducing agent. Several types of redox initiator pairs are known such as persulfite-bisulfite, persulfate-thiosulfate, persulfate-formaldehyde sulfoxylate, peroxide-formaldehyde sulfoxylate, peroxide-metallic ion (reduced), persulfate-metallic ion (reduced), benzoyl peroxide-benzene phosphinic acid, and benzoyl peroxide-amine wherein the amine acts as the reducing agent. The redox pair may be selected from any known redox pair such as a combination of benzoyl peroxide and dimethyl-p-toluidine, AMPS (ammonium persulfate) and TEMED (tetramethyl ethylene diamine), sulfur dioxide and tert-butyl hydroperoxide, potassium persulfate and acetone sodium bisulfite. In a specific aspect, the redox initiator pair is 1 wt % benzoyl peroxide with 1.5 wt % dimethyl-p-toluidine amine coinitiator.

In some embodiments, the initiator is a photoinitiator. The photoinitiator can be selected from one or more known photoinitiators. For example, the initiator can be selected from one or more of an alpha-hydroxyketone, an acyl phosphine oxide, a benzoyl peroxide with or without an amine co-initiator. Any known photoinitiator, or combination of one or more photoinitiators can be employed.

Photoinitiators may be employed alone or in combination including, but not limited to, DMPA (2,2-dimethoxy-2-phenylacetophenone), BDK (benzil dimethylketal), CPK (cyclohexylphenylketone), HDMAP (2-hydroxy-2-methyl-1-phenyl propanone), ITX (isopropylthioxanthrone), HMPP (hydroxyethyl-substituted alpha-hydroxyketone), MMMP (2-methyl-4′-(methylthio)-2-morpholinopropiophenone), BDMB (2-benzil-2-dimethylamino-1-(4-morpholinophenyl)-butanone-1), BP (Benzophenone), TPMK (methylthiophenyl-morpholinoketone), 4-Methylbenzophenone, 2-Methylbenzophenone, 1-Hydroxy cyclohexyl phenyl ketone, 2-Benzyl-2-(dimethylamino)-1-[4-(4-morpholinyl)phenyl]-1-butanone, Diphenyl Iodonium Hexafluorophosphate, Bis-(p-tolyl) iodonium hexafluorophosphate, 2-Methyl-1-[4-(methylthio)phenyl]-2-morpholinopropanone-1,2-Hydroxy-2-methyl-phenyl-propan-1-one, 1,7-bis(9-acridinyl) heptane, 2-Hydroxy-4′-hydroxyethoxy-2-methylpropiophenone, 2,2′-Bis(O-chlorophenyl-4,4′,5,′-tetraphenyl-1,2′-diimidazole, 9-Phenylacridine, N-phenylglycine, 2-(4-methoxyphenyl-4,6-bis(trichloromethyl)-1,3,5-triazine, p-toluene sulfonylamine, Tris-(4-dimethylaminophenyl) methane, Tribromomethyl phenyl sulfone, 2,4-Bis(trichloromethyl)-6-(p-methoxy) styryl-s-triazine, 2,4-Bis(trichloromethyl)-6-(3,4-dimethoxy) styryl-s-triazine, 4-(2-aminoethoxy)methyl benzophenone, 4-(2-hydroxyethoxy)methyl benzophenone, 2-Isopropylthioxanthone, 4-Isopropylthioxanthone, 4-Hydroxy benzophenone, 4-Methyl acetophenone, 4-(4-Methylphenylthiophenyl)-phenylmethanone, dimethoxyphenylacetophenone, camphorquinone, 1-Chloro-4-propoxythioxanthone, 2-Chlorothioxanthone, 2,2-Diethoxyacetophenone, 2,4-Diethylthioxanthone, 2-Dimethyl-aminoethylbenzoate, 2-Ethylhexyl-4-dimethylaminobenzoate, Ethyl 4-(dimethylamino)benzoate, 2-Isopropylthioxanthone, Methyl o-benzoyl benzoate, Methyl phenyl glyoxylate, 4,4′-Bis(diethylamino) benzophenone, 4-Phenylbenzophenone, 2,4,6- and Ethyl(2,4,6-trimethylbenzoyl)phenylphosphinate. In some embodiments, the photoinitiator can be selected from one or more acyl phosphine oxides such as BAPO (bis-acylphosphine oxide), phenyl-bis(2,4,6-trimethylbenzoyl)phosphine oxide, TPO (Diphenyl(2,4,6-trimethylbenzoyl)phosphine oxide), bis-trimethoxybenzoyl-phenylphosphine oxide, TPO-L, ethyl phenyl(2,4,6-trimethylbenzoyl)phosphinate, or MAPO (tris [1-(2-methyl) aziridinyl]phosphine oxide. The photoinitiator may be DMPA (2,2-dimethoxy-2-phenylacetophenone).

The initiator may be a commercially available BAPO bis-acyl phosphine oxide, for example, bis(2,4,6-trimethylbenzoyl)-phenylphosphineoxide (Omnirad 819, formerly known as Irgacure 819) from IGM Resins B.V, The Netherlands. The photoinitiator may be phenylbis(2,4,6-trimethylbenzoyl)phosphine oxide (Lucirin® TPO, BASF). The photoinitiator may be 1-hydroxy-cyclohexylphenyl ketone (OMNIRAD 184D, formerly Irgacure 184D) or 2-hydroxyl-2-methylpropiophenone (OMNIRAD 1173, formerly Irgacure 1173). In a specific embodiment, the photoinitiator is DMPA.

The polymerization photoinitiators are used in amounts effective to initiate polymerization in the presence of the curing radiation, typically about 0.01 to about 10 wt %, about 0.05 to about 7 wt %, about 0.1 to about 5 wt %, about 0.5 to 2 wt %, or about 1.2 to 1.9 wt % based on the total weight of the composition. In some embodiments, a low amount of photoinitiator may be employed, such as from 0.01 wt % to about 1%, about 0.05% to about 0.75 wt %, or about 0.1 wt % to about 0.5 wt %, or about 0.1 wt %. 0.2 wt %, or 0.3 wt %.

The photoinitiator composition can optionally further contain a coinitiator for example, EHA (2-ethyl hexylacrylate) or an amine coinitiator such as, for example, ethyl 4-(dimethylamino)benzoate, 2-ethylhexyl dimethylaminobenzoate, dimethylaminoethyl(meth)acrylate, or the like. Reactive amine polymerization coinitiators can be used, such as the coinitiator CN386 (a reactive amine adduct of tripropylene glycol diacrylate), commercially available from Sartomer, Darocure EHA, e.g., commercially available from Ciba, and the like. The coinitiator can be present in the composition in an amount of about 0.25 to about 20 wt %, specifically about 1 to about 10 wt %, and more specifically about 1 to about 5 wt %, based on the total weight of the composition.

The polymerizable compositions of the disclosure may be filled or unfilled. It has been unexpectedly found that unfilled urethane-acid copolymers according to the disclosure show lower wear rates than conventional filled urethane polymers, and dramatically lower wear rates than other unfilled acrylic polymers. In some embodiments, the compositions of the disclosure are unfilled. In some embodiments, the compositions of the disclosure comprise a filler.

### Fillers

In some embodiments, the polymerizable composition may include a filler. The ability to widely alter the filler loading without sacrifice to the strength and toughness makes the present invention well suited, for example, for use as a denture tooth material. The overall filler content also allows the modulus and surface hardness of the polymerized composite material to be altered with higher filler contents (especially when the OX50 nanofiller is included) leading to reduced wear rates. The filler content also aids in control of the coefficient of thermal expansion and is directly related to the x-ray opacity of the composite material.

There is no restriction in the type of filler that can be utilized in the filled compositions of the invention. In some embodiments, the filler material is selected from one or more of quartz, strontium, zirconium, and ytterbium-based particulate fillers. In some embodiments, the filler is selected from Ba glass, fumed silica, and ytterbium fluoride. In some embodiments, the filler phase is prepared from a bimodal mixture of barium glass with (Ba glass) and fumed silica (OX50). In some embodiments, the filler is ytterbium fluoride. In some embodiments, the filler employed in the filled polymer is Ba glass/OX50. In some embodiments, the filler is Ba glass/OX50/Yb. In some embodiments a mass ratio of 9:1 Ba glass/OX50 is employed. In some embodiments, the filler phase contains a silane methacrylate surface treatment (gamma-methacryloxypropyltrimethoxysilane. In some embodiments, the filler phase is prepared from a bimodal mixture of barium glass with methacrylate silane surface treatment (Ba glass) and fumed silica with methacrylate silane surface treatment (OX50). In some embodiments, the filler is ytterbium (Yb) glass with methacrylate silane surface treatment. In some embodiments, the filler is an ion leachable silicate glass. In some embodiments, the surfaces of the filler are coated with a surfactant. In some embodiments, the fillers comprise inert surfaces. In some embodiments, the fillers are surface modified with reactive coupling agents that allow their direct copolymerization with the resin matrix. In some embodiments, an OX50 nanofiller is employed. In some embodiments, filler is added in a range of between about 5 to about 90 wt %, about 10 to about 50 wt %, about 10 to about 20 wt %, about 50 to about 85 wt %; and about 70 to about 80 wt % with respect to the overall composite composition. In some embodiments one or more fillers is present, for example, at 50 wt % or lower, 25 wt % or lower, or 20 wt % or lower with respect to the overall composite composition for 3D printing applications. In some embodiments, one or more fillers is present at 75 wt % or higher compared to the weight of the filled composition. In some embodiments, one or more fillers is used at 85 wt % or higher compared to the weight of the filled composition.

The filler provides a dough-like consistency for the composite material in the monomeric state. The paste consistency can be raised or reduced depending on the choice of filler, ratio of the fillers and the filler loading level used. The optical properties of the paste and the final polymerized composite material depend on the degree of mismatch between the refractive indices of the fillers and the resin phase as well as the degree of conversion achieved during the polymerization process. A high degree of conversion (90% or higher or preferably 95% or higher) is desirable to maximize the mechanical properties of the polymeric material while minimizing or avoiding any leachable free monomer.

### Compositions

The compositions of the disclosure are suitable for 3D printing or molding of dental prosthetic devices and non-prosthetic appliances, denture bases and teeth, temporary restorations, splints, impression trays, surgical guides, casts, try-in set-ups, stents, and aligners. The compositions provided herein are also suitable for 3D printing of other devices and parts in the medical, automotive, communication, computer, electronics, and aeronautical industries.

Compositions are provided suitable for 3D printing comprising a tetra urethane (meth)acrylate monomer according to the disclosure. Homopolymers or copolymers may be formed from resin compositions provided herein. In some embodiments, a resin composition is provided comprising a tetraurethane (meth)acrylate monomer and an acidic monomer. Addition of the acidic monomer significantly decreases the viscosity of the polymerizable resin composition comprising the tetraurethane (meth)acrylate monomer, as shown in Table 1. Following photocure, the copolymers formed from the resin compositions comprising the tetraurethane (meth)acrylate monomer and the acidic monomer exhibit significantly increased flexural strength, flexural modulus and toughness compared to homopolymers formed from tetraurethane (meth)acrylate monomers alone, or conventional diurethane dimethacrylate monomers and MAA in a 1:1 acidic functionality to urethane functionality ratio.

The disclosure provides polymerizable compositions comprising a tetraurethane di(meth)acrylate monomer and an acidic monomer. Tetraurethane di(meth)acrylate and related monomers can be combined with an acidic comonomer reactive diluent in ratios of the acid to urethane functional groups that significantly exceed 1:1, which greatly reduces resin viscosity, but while also retaining or increasing the polymeric mechanical property enhancement found at a stoichiometric balance of acid to urethane groups. In some embodiments, the ratio of the acidic functionality from the acidic monomer to that of the urethane groups in the teraurethane di(meth)acylate monomer is in a ratio of from >1:1 up to 10:1 or higher, or from about 2:1 to about 10:1, 2:1, 3:1, 4:1, 5:1, 6:1, 7:1, 8:1, 9:1, or 10:1 that exhibit decreased viscosity, and can be used to produce polymers having comparable or increased flexural strength, flexural modulus, and toughness compared to a polymerizable composition comprising conventional diurethane dimethacrylate monomer UDMA and acidic monomer MAA in a 1:1 ratio of acidic to urethane functionalities, for example, as shown in Table 1. The tetraurethane di(meth)acrylate monomer may be a compound according to Formula (I).

In general, the TUD(M)A/acidic monomer resin composition polymers exhibit an improvement in flexural strength and toughness compared to UDMA/MAA polymers, as shown in Table 1. Surprisingly, when the acid to urethane functional group ratio is increased to 2:1 (this corresponds to 8 moles of methacrylic acid (MAA) with 1 mole of TUD(M)A), the mechanical strength properties are not significantly affected compared with the copolymer with a 1:1 stoichiometric balance of the acid-urethane functional groups, while simultaneously, the formulation viscosity is dramatically reduced to the low viscosity state. This demonstrates that low viscosity formulations based on these multi-urethane monomers can be obtained without sacrifice of the exceptional polymeric mechanical properties. This finding is in contrast to prior published study by Tanaka et al. (Dental Materials Journal 2001; 20:206-15) which shows a precipitous reduction in mechanical strength for a UDMA/MAA formulation taken beyond the 1:1 acid to urethane functional group ratio.

The strength values with TUD(M)A/acidic comonomer resins are higher than any achieved to date with any other acid-reinforced urethane (meth)acrylate compositions. The TUD(M)A monomers with varied proportions of acidic comonomer result in various viscosity states including high viscosity (homomonomer), which presents only modest mechanical properties, moderate viscosity (1:1 acid/urethane comonomer) and low viscosity (>1:1 acid/urethane comonomer) with the copolymeric materials found to out-perform other photopolymeric materials.

The disclosure provides polymerizable compositions comprising a diurethane di(meth)acrylate monomer, a monourethane mono(meth)acrylate or a monourethane di(meth)acrylate, and optionally an acidic monomer.

In some embodiments, compositions are provided comprising low viscosity mono-urethane (meth)acrylate monomers as a reactive diluent with high viscosity di-urethane (meth)acrylate or multi-urethane (meth)acrylate monomers without the acidic comonomer. The resin viscosity reduction may be achieved without reducing the overall urethane group concentration.

The diurethane di(meth)acrylate monomer may be any suitable diurethane di(meth)acrylate monomer. The diurethane di(meth)acrylate monomer may be a PCL diurethane di(meth)acrylate monomer. The diurethane di(meth)acrylate monomer may be a conventional diurethane di(meth)acrylate monomer such as UDMA. The monourethane mono(meth)acrylate may be a compound according to Formula (II). The monourethane di(meth)acrylate may be a compound according to Formula (IIIa) or Formula (IIIb).

The molar ratio of the diurethane di(meth)acrylate monomer to the monourethane mono(meth)acrylate monomer or the monourethane di(meth)acrylate monomer is from about 90:10 to about 50:50; about 80:20 to about 50:50; about 70:30, or about 50:50.

The acidic monomer may be present in the composition in an excess of the acidic functionality to that of the urethane groups in a ratio of from >1:1 up to 10:1 or higher, or from about 2:1 to about 10:1, 2:1, 3:1, 4:1, 5:1, 6:1, 7:1, 8:1, 9:1, or 10:1 that exhibit decreased viscosity, and can be used to produce polymers having comparable or increased flexural strength, flexural modulus, and toughness compared to a polymerizable composition comprising conventional diurethane dimethacrylate monomer UDMA and acidic monomer MAA in a 1:1 ratio of acidic to urethane functionalities (Table 3).

The high viscosity of UDMA as a common di-urethane dimethacrylate monomer can be dramatically reduced by addition of one of these mono-urethane comonomers.

In Table 5, the viscosity and polymer properties are provided for polymerizable resin formulations of UDMA with either mono-urethane mono(meth)acrylates or mono-urethane di(meth)acrylates, as a means to control both the pre-cure viscosity and the covalent network density in the copolymers without significantly reducing the overall urethane group concentration. Relative to the UDMA homopolymer and the UDMA/MAA copolymer mechanical properties (shown in Table 2), the copolymers made with UDMA and 30 mol % of mono-urethane monomethacrylates, such as IEM-BzOH and IEM-PEOH, are comparable or better in terms of strength and modulus in the presence of MAA while raising the mono-urethane monomethacrylate to 50% dropped the strength but increased the modulus. With 50 mol % of the IEM-HEMA mono-urethane dimethacrylate used with UDMA, a very low viscosity resin is obtained that also produces polymeric strength and modulus results with MAA reinforcement that are improved relative to the UDMA/MAA formulation. The important aspect is that the commercially available UDMA can be leveraged here for a significant portion of the formulation that gives excellent polymeric mechanical properties while the blending of multi- and mono-urethane monomers provides resin viscosities well below the 100 mPa·s threshold along with the ability to maintain a high overall urethane group content. As shown in Table 5, the enhancement in the ambient real-time conversion and post-cure conversion seen for the formulations that included AA as either the only acid-functional comonomer or as an equimolar mixture of AA and MAA with the acid to urethane group ratio fixed at 1:1, again demonstrates the ability of AA to facilitate the photopolymerization of urethane methacrylates while maintaining the polymeric mechanical properties that are obtained with the MAA diluted and reinforced analog formulations. Also, the among the isomers of IEM-PhOH and PhNCO-HEMA that were both solids either with or without added MAA, these both mixed with UDMA to give homogeneous resins where the PhNCO-HEMA was significantly more effective in lowering the viscosity of UDMA and simultaneously providing good polymeric mechanical properties relative to the IEM-PhOH.

### Polymerization

The polymerizable resin compositions of the disclosure may be polymerized by any appropriate method. The polymerization of these materials may be assisted by light, pressure and/or heat to maximize their conversion and properties. The polymerizable resin compositions of the disclosure may be photocured. For example, the polymerizable compositions of the disclosure may be subjected to ambient photopolymerization in the presence of a photoinitiator using medium pressure mercury arc UV light with 365 nm filter to give an incident irradiance of 100 mW/cm².

The post-cure process may be an important component of the overall polymer production process. The post-cure can improve strength and toughness, as well as increase the final level of conversion achieved. The post-cure process may comprise exposure to different light and/or thermal treatment and times. For example, the polymer and copolymers may be post-cured in a commercial light-curing oven, for example, for 1 hour at 80° C. with concurrent exposure to 365 and 405 nm light sources.

The polymerization efficiency may be monitored, for example, ambient conversion values may be reported after a period of time under continuous light exposure using real-time near-infrared spectroscopy to monitor the =CH₂ (meth)acrylate reactive group at ˜6165 cm⁻¹.

### Methods of Using the Polymerizable Compositions

A method is provided for creating a two-dimensional film or a three-dimensional shaped part comprising molding, free-form fabricating, or printing of a polymerizable resin composition according to the present disclosure. The film or shaped part may be a prosthetic device or a non-prosthetic appliance. The prosthetic device or non-prosthetic appliance may be a medical or dental device or appliance. A method of preparing a shaped part is provided comprising 3-dimensional (3D) printing of a polymerizable resin composition according to the present disclosure. In some embodiments, a method for providing one-step molded parts is provided, which can be polymerized by heat, redox or light. This may be applied to 2D films as well as 3D parts. Regarding the photopolymerization-based 3D printing process, the 3D part may be constructed in either a continuously formed or sequentially layered 3D printing process that results from the spatially structured photopolymerization applied either continuously or sequentially to create each layer. Either way, the part may be photocured during the entire building process and optionally subjected to post-polymerization cure applied at the end. In some embodiments, the extent of polymerization may be sufficient to allow the printed part along with any supporting structure to be self-supporting. Optionally, final polymerization (post-cure) may then be used as needed to complete the processing of the part.

A method of preparing a shaped part is provided comprising 3-dimensional (3D) printing of a polymerizable resin composition according to the present disclosure to form a shaped part; and polymerizing the shaped part. The printed part may be subjected to post-cure treatment. The shaped part may be a dental prosthetic device or dental non-prosthetic appliance. The dental prosthetic device may be a crown, bridge, denture, implant, or other prosthetic device. The dental non-prosthetic appliance may be an aligner, dental splint, retainer, mouthguard, whitening tray, or other intraoral appliance. For example, the dental non-prosthetic appliance may fit over existing teeth instead of taking the place of missing tissue. The shaped part may be a biomedical part such as for use in bone repair, cardiovascular stents, or other biomedical part. Methods for use of the compositions of the disclosure may include dental applications, biomedical applications, or non-dental, non-medical applications such as, for example, an automotive, aerospace, microelectronics, electrical, plumbing, or other applications.

A method of preparing a shaped part such as a dental appliance or dental prosthetic device is provided comprising: dispensing a polymerizable resin composition of the disclosure; shaping the mixture into the form of the shaped dental prosthetic device; and optionally photopolymerizing the shaped mixture. The dental appliance may be a dental aligner appliance, bite splint, retainer, whitening tray, or other dental appliance. The dental prosthetic device may be a crown, bridge, denture, implant, or other prosthetic device.

A two-dimensional film or a three-dimensional shaped part is provided comprising a polymer created from the polymerization of the polymerizable resin composition according to the disclosure.

A two-dimensional film or a three-dimensional shaped part is provided comprising a polymer created from the polymerization of the polymerizable resin composition according to the disclosure in admixture with one or more fillers.

In some embodiments, a dental prosthetic device is provided comprising a polymer created from the polymerization of the resin according to the disclosure in admixture with one or more fillers.

A dispensing device is provided comprising an unpolymerized quantity of a polymerizable composition comprising a tetraurethane (meth)acrylate monomer, and an acidic monomer; or a diurethane di(meth)acrylate monomer, a monourethane mono(meth)acrylate or a monourethane di(meth)acrylate, and optionally an acidic monomer. In some embodiments, the composition comprises one or more fillers.

A polymerizable composition is provided comprising: particles of filler, a tetraurethane (meth)acrylate monomer, and an acidic monomer. A polymerizable composition is provided comprising: particles of filler, a diurethane di(meth)acrylate monomer, a monourethane mono(meth)acrylate or a monourethane di(meth)acrylate, and optionally an acidic monomer. In some embodiments, the optional filler may be present at 0-25 wt %, 2-20 wt % or 5-15 wt % of the total material weight. In some embodiments, a filler may be present at from 25-95 wt %, 30-92 wt %, 40 wt % to 90 wt %; 50 wt % to 85 wt %; or 70 wt % to 80 wt % of the total material weight.

A dispensing device is provided comprising an unpolymerized quantity of a polymerizable composition comprising a tetraurethane (meth)acrylate monomer, and an acidic monomer. A dispensing device is provided comprising an unpolymerized quantity of a diurethane di(meth)acrylate monomer, a monourethane mono(meth)acrylate or a monourethane di(meth)acrylate, and optionally an acidic monomer. In some embodiments, the composition comprises one or more fillers.

The following examples are given to illustrate, but not limit, the scope of this invention. Unless otherwise indicated, all parts and percentages are by weight. Unless otherwise indicated, all numbers expressing quantities of ingredients, properties such as molecular weight, reaction conditions, and so forth used in the specification and claims are to be understood as being modified in all instances by the term “about.” The term “about” represents +/−10% of the numerical term to which it is applied. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

### Examples

Polymer flexural strength (MPa), flexural modulus (GPa), and toughness (J/m³) were calculated using a 3-point flexural test, carried out with a hydraulic universal test system (858 Mini Bionix, MTS Systems Corporation, Eden Prairie, MN, USA) using a span width of 20 mm and a crosshead speed of 1 mm/min. The flexural strength (FS, σ) in MegaPascals (MPa) and flexural modulus (modulus, E_{f}) in GigaPascals (GPa) were calculated using the following equations:

$$σ = \frac{3 F l}{2 b {h}^{2}} \qquad ( Equation 2 )$$

$${E}_{f} = \frac{{F}_{1} {l}^{3}}{4 b {h}^{3} d} \qquad ( Equation 3 )$$

where F is the peak load (in N), 1 is the span length (in mm), b is the specimen width (in mm), h is the specimen thickness (in mm); and d is the deflection (in mm) at load F1 (in N) during the straight line portion of the trace according to ISO/DIS 4049:2019). ISO/DIS 4049 is the international standard for “Dentistry—Polymer-based filling, restorative and luting materials”. Flexural strength test is one of the tests specified in this standard for the polymer-based filling, restorative and luting materials. Unless otherwise specified, samples for mechanical strength and other tests were tested on approximately 5-8 specimens per sample (approximately 25 mm×2 mm×2 mm).

Fracture toughness K_{IC} describes the resistance of a material to crack propagation. The higher the value of the critical stress intensity factor (K_{IC}[MPa√m]), the better the prognosis for long term clinical behavior of the material. The single-edge notched-beam fracture toughness test, ASTM D5045 may be employed to determine fracture toughness K_{IC}.

Unless otherwise specified, ambient photopolymerization is carried out under the following conditions: 0.1 wt % 2,2-dimethoxy-2-phenylacetophenone (DMPA) photoinitiator; medium pressure mercury arc UV light with 365 nm filter to give an incident irradiance of 100 mW/cm². The polymer and copolymers were then also post-cured in a commercial light-curing oven for 1 hour at 80° C. with concurrent exposure to 365 and 405 nm light sources.

Near-Infrared spectroscopy (NIR) was performed on a Nicolet Nexus 670 to analyze degree of conversion during or following polymerization. Unless otherwise specified, ambient conversion values are reported after 10 minute continuous light exposure using real-time near-Infrared spectroscopy to monitor the =CH₂ (meth)acrylate reactive group at ˜6165 cm⁻¹.

Proton Nuclear Magnetic Resonance (¹H-NMR) can be used to confirm structural identity, and to integrate, thus quantify, protons of interest (Varian 300 MHz; performed in CDCl₃). For example, CH₂ protons, CH₂OCH₂ protons, and CH₃ protons may be assigned at about δ 1.92, 3.75-60, and 0.89 ppm chemical shifts, respectively, and may be integrated to determine relative abundance. 1H-NMR may also be employed to determine average PCL (n) values.

Viscosity may be measured by any appropriate test method. In some embodiments, viscosity of polymerizable resin compositions may be measured by ASTM D2857 or ASTM D5225.

### Example 1A. Tetraurethane Di(Meth)Acrylate [TUD(M)A]Monomers

Tetraurethane di(meth)acrylate [TUD(M)A]monomers according to Formula (I) were prepared with various core structures provided by different diamine R groups, as well as variations in the methacrylate (X=—CH₃) or acrylate (X=—H) functionality, and in the spacer group length (n=1 or 2) between the reactive group and the external urethane group, as shown in FIG. 1A.

For example, tetra-urethane dimethacrylate monomers (TUD(M)Ala and TUD(M)A 1b; FIG. 1) were prepared by the two-step reaction of a diamine (e.g., 2,2,4-trimethylhexane-1,6-diamine) with two equivalents of ethylene carbonate to make the internal urethane linkages, followed by addition of either isocyanatoethyl methacrylate (IEM) or isocyanatoethoxyethyl methacrylate (IEMEG) to prepare the tetra-urethane dimethacrylate monomers of Formula (I), 1a and 1b, respectively as shown in FIG. 1A.

The corresponding core structures 1-7 according to Formula (I) were derived from diamines as shown in FIG. 1A, such as 2,2,4-trimethylhexane-1,6-diamine and 2,4,4-trimethylhexane-1,6-diamine (in 1); 1,2-diaminoethane (in 2); 1,3-diaminopropane (in 3); 1,2-bis(2-aminoethoxy) ethane (in 4); 2-methyl-1,5-diaminopentane (in 5); isophorone diamine (in 6); and m-xylylenediamine (in 7) as shown in FIG. 1A. The room temperature (˜23° C.) viscosities of these TUD(M)A monomers were measured along with the formulations of TUD(M)A with either acrylic acid (AA) or methacrylic acid where the proportions of the acid to urethane functional group ratio were 1:1 or 2:1 (x2) as shown in Table 1. These monomers and comonomers were photocured and run through a thermal/photo post-cure process before subjecting the polymers to mechanical testing in three-point bending mode with the results listed in Table 1.

TABLE 1
TUDMA Polymerizable Composition Viscosities, and Polymer
Flexural Strength, Flexural Modulus, and Toughness

| | Viscosity, | Flexural | Flexural | Toughness, |
|---|---|---|---|---|
| Monomer | mPa · s | strength, MPa | modulus, GPa | J/m³ |
| UDMA | 8912 ± 104 | 157.4 ± 6.2 | 3.13 ± 0.12 | 9.2 ± 1.6 |
| UDMA/MAA | 117 ± 2.7 | 179.7 ± 25.0 | 4.14 ± 0.31 | 9.3 ± 6.4 |
| TUDMA1a | 12,836 ± 2783 | 86.5 ± 4.3 | 2.10 ± 0.21 | 6.8 ± 3.8 |
| TUDMA1a/MAA | 264.9 ± 27.0 | 214.0 ± 25.6 | 4.86 ± 0.29 | 8.2 ± 3.7 |
| TUDMA1a/MAA | ″ | 191.0 ± 8.9 | 4.23 ± 0.18 | 18.0 ± 9.8 |
| (wet)* | | | | |
| TUDMA1a/MAA(x2)^{†} | 39.2 ± 3.9 | 212.5 ± 18.8 | 5.15 ± 0.28 | 5.9 ± 1.4 |
| TUDMA1b | 5212 ± 984 | | | |
| TUDMA1b/MAA | 246.8 ± 54.3 | 258.6 ± 12.0 | 4.94 ± 0.12 | 12.0 ± 2.2 |
| TUDMA1b/MAA(x2)^{†} | 29.5 ± 4.1 | | | |
| TUDMA1a/AA | 254.3 ± 26.1 | 250.2 ± 8.6 | 5.18 ± 0.17 | 19.1 ± 7.5 |
| TUDMA1a/AA(x2)^{†} | 35.4 ± 4.5 | | | |
| TUDMA1b/AA | 249.9 ± 37.1 | | | |
| TUDMA1b/AA(x2)^{†} | 40.5 ± 3.9 | 214.2 ± 20.2 | 4.77 ± 0.37 | 24.9 ± 12.3 |
| TUDA1c | 5458 ± 264 | | | |
| TUDA1c/AA | 145.4 ± 17.2 | | | |
| TUDA1c/AA(x2)^{†} | 37.1 ± 4.9 | 206.0 ± 12.2 | 5.15 ± 0.32 | 23.0 ± 9.3 |
| TUDMA4/MAA | n/a | 248.6 ± 13.6 | 5.12 ± 0.26 | 16.6 ± 9.4 |
| TUDMA5/MAA | n/a | 209.0 ± 34.8 | 6.31 ± 0.15 | 4.1 ± 1.4 |
| TUDMA7/MAA | n/a | 223.0 ± 39.2 | 5.58 ± 0.52 | 6.0 ± 2.23 |

*Equilibrated water uptake = 1.94 ± 0.15 wt %
^{†}Acid to urethane functional group ratio of 2 (x2); otherwise, the acid:urethane ratio is 1:1

In general, the TUDMA/acidic monomer resin composition polymers exhibit an improvement in flexural strength and toughness compared to UDMA/MAA polymers. Surprisingly, when the acid to urethane functional group ratio is increased to 2:1 (this corresponds to 8 moles of methacrylic acid (MAA) with 1 mole of TUD(M)A), the mechanical strength properties are not significantly affected compared with the copolymer with a 1:1 stoichiometric balance of the acid-urethane functional groups, while simultaneously, the formulation viscosity is dramatically reduced to the low viscosity state. This demonstrates that low viscosity formulations based on these urethane oligomers can be obtained without sacrifice of the exceptional polymeric mechanical properties. This finding is in contrast to prior published study by Tanaka et al. (Dental Materials Journal 2001; 20:206-15) which shows a precipitous reduction in mechanical strength for a UDMA/MAA formulation taken beyond the 1:1 acid to urethane functional group ratio.

The strength values with TUD(M)A/acidic comonomer resins are higher than any achieved to date with any other acid-reinforced urethane (meth)acrylate compositions. Reinforced TUD(M)A monomers in various viscosity states including high viscosity (homomonomer), moderate viscosity (1:1 acid/urethane comonomer) and low viscosity (>1:1 acid/urethane comonomer) states have been found to out-perform other photopolymeric materials.

### Example 1B. Additional multi-urethane (meth)acrylate monomers

Additional synthetic routes for production of multi-urethane (meth)acrylate monomer compounds are outlined in FIG. 1B. The mono-amine R—NH₂ is exposed to 1,3-dioxolan-2-one to obtain the 2-hydroxyethyl carbamate intermediate. The intermediate is treated with IEM, IEMEG, or IEA isocyanates to obtain a di-urethane mono(meth)acrylate [DUM(M)A]. A similar reaction scheme employing a di-amine R((NH₂)₂ may be employed to obtain TUD(M)A. Use of a tri-amine R((NH₂)₃ or tetraamine R((NH₂)+gives the hexa-urethane tri(meth)acrylate [HUT(M)A], octa-urethane tetra(meth)acrylate [OUT(M)A]monomer compounds, respectively.

The diamine may be a poly(ethylene glycol)diamine according to

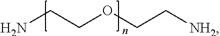

wherein n=1-20, 2-12, or 1, 2, 3, 4, 5, 6, 7, 8, 9, 10, 11 or 12. These types of poly(ethylene glycol)diamine compounds are commercially available, for example, from Sigma-Aldrich chemical company. The diamine may be a poly(propylene glycol)diamine according to

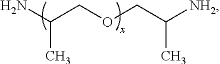

wherein x may be 2-70, or about 2-12. These types of poly(propylene glycol)diamine compounds are commercially available, for example, as JEFFAMINE® D series compounds form, for example Sigma-Aldrich chemical company.

### Example 2. Compositions comprising polycaprolactone (PCL)diurethane (meth)acrylate monomers with Acrylic Acid

This example shows that viscosities of compositions comprising PCL diurethane (meth)acrylate monomers and acidic monomers can be reduced with an excess of the acidic functionality to that of the urethane groups.

A representative PCL diurethane (meth)acrylate is shown in FIG. 4. This PCL diurethane (meth)acrylate monomer, in which m+n˜16, was prepared from a polycaprolactone (PCL)-diol with molecular weight of approximately 2000 g/mol that is first reacted with two-equivalents of IEM to produce the di-urethane dimethacrylate with two extended oligomeric PCL segments. Preparation of the PCL monomer was performed according to in U.S. 63/105,068. Previously this PCL diurethane (meth)acrylate was only used with MAA. A composition comprising the PCL monomer of FIG. 4 and AA was found to produce a significantly lower viscosity comonomer mixture. When this PCL monomer is formulated with a 10-fold functional group excess of AA acid to urethane functional groups, a very low viscosity (8.58±0.38 mPa·s) resin is obtained.

In addition to the urethane-acid interaction, the acidic functionality apparently interacts in a physically reinforcing positive manner with the polyester linkages of the PCL groups of the PCL monomer such that a very mechanically strong and robust copolymer is produced: flexural modulus 3.11±1.07 GPa; flexural strength 118.4±24.4 MPa; and a surprisingly high toughness of 20.4±3.3 J/m³.

### Example 3. Monourethane Mono(Meth)Acrylate Monomers

A variety of mono-urethane mono-(meth)acrylate monomers were synthesized using highly efficient alcohol-isocyanate reactions as follows. However, other non-isocyanate routes to these monomers are available.¹⁴⁻¹⁶

Structures of the synthesized mono-urethane mono(meth)acrylate monomers are provided in FIG. 6. The monomers presented here were obtained by stoichiometric reaction of isocyanatoethyl methacrylate (IEM; FIG. 7) with a variety of alkyl and aryl alcohols. Structural variation was achieved by use of the ethylene glycol extended methacrylate-functionalized isocyanate (IEMEG) as shown for the reaction product of IEMEG and benzyl alcohol (BzOH). Alternatively, the mono-urethane monomers can be prepared from HEMA or related hydroxy methacrylates by reaction with an alkyl or aryl isocyanate. This approach demonstrates that isomeric monomer pairs that differ only in the configuration of the urethane group with respect to the polymerizable (meth)acrylate group are easily achieved as indicated by the IEM-BzOH and HEMA-BzNCO as well as IEM-BuOH and HEMA-BuNCO monomers. Replacement of the methacrylate groups with acrylate functionality is easily achieved by substitution of isocyanatoethyl acrylate (IEA) in place of IEM or by use of 2-hydroxyethyl acrylate (HEA) in place of HEMA. There are numerous further examples of mono-urethane monomers that could be constructed in this manner.

The monomers displayed in FIG. 6 are all liquid at room temperature and their bulk viscosities are given in Table 2.

TABLE 2
Ambient viscosity of neat mono-urethane mono-methacrylates
and their mixtures with methacrylic acid (MAA)
at a 1:1 acid to urethane ratio.

| | Molecular | | Viscosity with |
|---|---|---|---|
| | weight, | Viscosity, | added MAA, |
| Monomer | g/mol | mPa · s | mPa · s |
| UDMA | 470.6 | 8912 | 117.6 ± 2.7 |
| IEM-BzOH | 263.3 | 31.0 ± 3.2 | 11.8 ± 1.5 |
| IEMEG-BzOH | 354.4 | 30.2 ± 6.5 | 14.0 ± 2.1 |
| BzNCO-HEMA | 263.3 | 36.4 ± 0.4 | 13.3 ± 2.2 |
| IEM-BuOH | 229.3 | 14.1 ± 1.0 | 4.9 ± 1.2 |
| IEM-PEOH | 293.3 | 37.4 ± 2.1 | 13.5 ± 1.4 |
| IEM-IBOH | 309.4 | 413.7 ± 17.0 | 87.6 ± 5.0 |
| IEM-Triton45 | ~581.7 | 316.5 ± 35.9 | 213.6 ± 20.2 |
| IEM-PhOH | 249.3 | solid | solid |
| PhNCO-HEMA | 249.3 | solid | solid |

These monomers are dramatically lower in viscosity compared with a conventional di-urethane dimethacrylate (UDMA; prepared by the reaction of 2,2,4-(2,4,4)-trimethylhexyldiisocyanate with two equivalents of 2-hydroxyethyl methacrylate), which is considered to have a relatively low viscosity among urethane monomers at ˜8900 mPa·s, and certainly lower than that of reactive urethane oligomers, which are typically measured at elevated temperatures because of the excessively high viscosity values involved.

For the varied mono-urethane mono-methacrylate monomers reported here, the ambient bulk viscosities are low, but they also cover a significant range at the low end of the viscosity scale. For example, whereas benzyl methacrylate, which lacks any hydrogen bond donor groups, has a viscosity of ˜3 mPa·s, the viscosity of the benzyl urethane methacrylate is about an order of magnitude greater in viscosity due to both hydrogen bond donor and acceptor character associated with the urethane linkage. It is somewhat surprising that the isobornyl urethane methacrylate is approximately 50-fold more viscous than isobornyl methacrylate (˜8 mPa·s), which suggests the sterically demanding isobornyl groups interfere with simple urethane-urethane dimer formation and instead favor the more extended hydrogen bonding that promotes higher viscosity. The amphiphilic structure of the Triton45 also leads to a higher viscosity mono-urethane monomer likely due to the hydrogen bonding interaction between the urethane and the extended ether groups. The IEM-BuOH monomer may well be the lowest viscosity bulk urethane (meth)acrylate monomer possible because the analog prepared with IEM and ethyl alcohol is a crystalline solid at room temperature.

### Example 4. Monourethane di(meth)acrylate Monomers

The extension of the mono-urethane mono-(meth)acrylates, which produce linear polymers, to a crosslinkable version that retains the single urethane group between two (meth)acrylate functional groups was also pursued.

A useful series of mono-urethane di(meth)acrylates was prepared from the pairwise combination of IEM or IEA with HEMA or HEA, as shown in FIG. 7. These four mono-urethane di(meth)acrylate monomers, as well as several additional variations that can similarly be obtained by using extended versions of either or both the isocyanato (meth)acrylate or the hydroxy (meth)acrylate components, as represented in FIG. 8.

Branched crosslinkers can readily be obtained by using hydroxy(meth)acrylates such as 2-hydroxy-3-phenoxypropyl(meth)acrylate (HPPMA), hydroxypropyl(meth)acrylate (HPMA) and hydroxybutyl(meth)acrylate (HBMA) in reactions with (meth)acrylate-functional isocyanates. With regard to branched urethane monomer structures, we also investigated the placement of the mono-urethane group in a side-chain off the crosslinker unit of a di(meth)acrylate. Through use of glycerol dimethacrylate (GDMA) with either butyl isocyanate (BuNCO) or phenyl isocyanate (PhenylNCO), the side-chain urethanes were prepared as shown in FIG. 9. However, both the mono-urethane di(meth)acrylate homopolymers and the copolymers with one molar equivalent of added MAA offered low mechanical properties and were not pursued further as low viscosity materials. Therefore simple presence of urethane and polymerizable groups does not guarantee positive results.

The monomeric and polymeric physical and mechanical properties of the exemplary series of synthesized mono-urethane di(meth)acrylates is shown in Table 3. Tensile properties of mono-urethane di(meth)acrylate polymers and copolymers are shown in Table 4.

Unless otherwise specified, copolymers of the mono-urethane di(meth)acrylates and MAA were prepared as equimolar mixtures.

The neat room temperature viscosities are provided along with the degree of conversion achieved with an ambient photopolymerization under the following conditions: 0.1 wt % 2,2-dimethoxy-2-phenylacetophenone (DMPA) photoinitiator; medium pressure mercury arc UV light with 365 nm filter to give an incident irradiance of 100 mW/cm². The ambient conversion values are reported after 10 minute continuous light exposure using real-time near-infrared spectroscopy to monitor the =CH₂ (meth)acrylate reactive group at ˜6165 cm⁻¹. The polymer and copolymers were then also post-cured in a commercial light-curing oven for 1 hour at 80° C. with concurrent exposure to 365 and 405 nm light sources.

All the polymeric mechanical properties are reported for post-cured materials based on 3-point bend testing of 2×2×25 mm specimens (n=5-8) on a 20 mm span with a crosshead speed of 1 mm/min. The tensile properties of two of the mono-urethane dimethacrylate homopolymers and the corresponding MAA-diluted (1:1 molar) are shown in Table 4.

TABLE 3
Physical and mechanical properties of mono-urethane di(meth)acrylate
monomers, homopolymers and copolymers

| | | Polymer properties | | | | |
|---|---|---|---|---|---|---|
| | | Conversion, % | | Flex | Flex | |
| Monomer or | Viscosity, | Ambient | Post | strength, | modulus, | Toughness, |
| comonomer | mPa · s | photocure | cure | MPa | GPa | J/m³ |
| UDMA | 8912 ± 104 | * | * | 157.6 ± 6.5 | 3.13 ± 0.12 | 2.17 ± 1.38 |
| “+MAA | 118 ± 3 | | | 179.7 ± 25.0 | 4.14 ± 0.31 | 2.91 ± 1.06 |
| IEM-HEMA | 27.9 ± 2.1 | 58.2 ± 1.3 | 75.4 ± 1.6 | 135.5 ± 21.0 | 3.52 ± 0.23 | 3.59 ± 1.35 |
| “+MAA | 13.9 ± 1.0 | 68.5 ± 1.0 | 87.7 ± 1.1 | 163.8 ± 28.3 | 4.24 ± 0.25 | 4.96 ± 1.84 |
| IEMEG-HEMA | 30.6 ± 1.9 | 74.2 ± 2.0 | 91.5 ± 0.7 | 113.4 ± 11.7 | 2.84 ± 0.22 | 2.94 ± 0.73 |
| “+MAA | 15.8 ± 1.1 | 69.5 ± 1.6 | 84.8 ± 0.7 | 181.2 ± 16.5 | 3.70 ± 0.16 | 8.72 ± 2.77 |
| IEM-HEA | 53.3 ± 5.5 | 75.3 ± 2.5 | 82.9 ± 1.1 | 158.0 ± 23.8 | 4.14 ± 0.21 | |
| “+MAA | 15.9 ± 1.2 | 70.8 ± 1.9 | 82.1 ± 0.2 | 154.0 ± 42.9 | 4.88 ± 0.33 | |
| IEA-HEMA | 42.2 ± 5.8 | 78.3 ± 2.1 | 87.6 ± 0.1 | 157.7 ± 19.2 | 3.89 ± 0.27 | |
| “+MAA | 12.3 ± 1.8 | 69.2 ± 1.3 | 81.6 ± 1.1 | 170.7 ± 32.8 | 4.25 ± 0.36 | |
| IEA-HEA | 70.0 ± 9.9 | 89.6 ± 0.1 | 92.7 ± 2.2 | 155.2 ± 20.5 | 3.75 ± 0.13 | |
| “+MAA | 17.9 ± 2.4 | 83.8 1.7 | 86.6 ± 0.1 | 173.2 ± 30.7 | 4.47 ± 0.27 | |
| IEM-HEMAC1 | 349.5 ± 45.5 | 69.8 ± 1.0 | 89.7 ± 1.5 | 138.6 ± 23.1 | 3.22 ± 0.19 | 5.48 ± 3.35 |
| “+MAA | 77.6 ± 5.8 | 70.7 ± 2.0 | 90.4 ± 0.5 | 181.4 ± 21.5 | 4.02 ± 0.22 | 7.45 ± 3.74 |
| “+MAA(1:1.3) | — | — | — | 164.2 ± 14.5 | 3.88 ± 0.14 | 4.51 ± 1.71 |
| “+MAA(1:1.5) | — | — | — | 169.1 ± 24.5 | 3.96 ± 0.11 | 7.34 ± 4.90 |
| IEMEG- | — | 87.3 ± 1.4 | 98.0 ± 0.5 | 101.9 ± 13.2 | 2.53 ± 0.17 | 4.00 ± 2.74 |
| HEMAC1 | — | 81.9 ± 1.7 | 96.0 ± 0.3 | 146.9 ± 19.3 | 3.38 ± 0.27 | 8.03 ± 3.74 |
| “+MAA | | | | | | |
| IEM-E5HEMA | — | — | — | — | — | |
| “+MAA | 38.3 ± 7.1 | 97.2 ± 0.6 | — | 26.8 ± 1.2 | 0.66 ± 0.04 | |
| IEM-HPPMA | 141.3 ± 10.8 | 69.2 ± 1.6 | 88.9 ± 0.1 | 117.3 ± 13.6 | 3.58 ± 0.17 | |
| “+MAA | 26.3 ± 2.0 | 67.9 ± 1.6 | 87.9 ± 1.3 | 138.6 ± 16.3 | 4.11 ± 0.28 | |
| IEMEG- | 90.1 ± 6.6 | 75.7 ± 1.3 | 91.4 ± 0.1 | 108.5 ± 14.6 | 3.37 ± 0.13 | |
| HPPMA | 37.5 ± 1.8 | 74.3 ± 1.5 | 90.5 ± 0.2 | 136.1 ± 20.9 | 4.39 ± 0.24 | |
| “+MAA | | | | | | |
| IEM-HPMA | 81.5 ± 5.8 | 57.9 ± 0.1 | 78.9 ± 0.1 | 119.5 ± 20.6 | 3.50 ± 0.12 | |
| “+MAA | 26.4 ± 3.8 | 58.3 ± 1.2 | 78.6 ± 1.3 | 125.2 ± 45.0 | 4.03 ± 0.26 | |
| IEM-HBMA | 91.2 ± 3.5 | 59.2 ± 0.1 | 81.5 ± 0.1 | 109.5 ± 29.0 | 3.08 ± 0.19 | |
| “+MAA | 28.4 ± 2.2 | 55.8 ± 4.1 | 78.5 ± 1.3 | 134.9 ± 26.7 | 3.47 ± 0.21 | |

TABLE 4
Tensile properties of mono-urethane di(meth)acrylate
polymers and copolymers

| | Tensile | Tensile | Tensile |
|---|---|---|---|
| Monomer or | strength, | modulus, | toughness, |
| comonomer | MPa | GPa | J/m³ |
| IEM-HEMA | 60.4 ± 16.2 | 2.04 ± 0.03 | 1.93 ± 0.25 |
| ″ + MAA | 69.8 ± 12.5 | 2.93 ± 0.07 | 1.15 ± 0.37 |
| IEMEG-HEMA | 47.8 ± 7.2 | 1.79 ± 0.12 | 0.92 ± 0.25 |
| ″ + MAA | 78.8 ± 8.0 | 2.26 ± 0.13 | 1.74 ± 0.15 |

### Example 5. Addition of Mono-Urethane Mono(Meth)Acrylate or Mono-Urethane Di(Meth)Acrylate Monomers to a Conventional Urethane Monomer

The high viscosity of UDMA (8912 mPa·s) as a common di-urethane dimethacrylate monomer can be dramatically reduced by addition of one of the mono-urethane comonomers, and further reduced by addition of an acidic monomer. Table 5 shows properties of mono-urethane (meth)acrylates combined with UDMA, and when further combined with an acidic monomer. Values in parentheses in Table 5 indicate molar ratio of UDMA to mono-urethane component.

TABLE 5
Properties of mono-urethane (meth)acrylates combined with UDMA

| | | Polymer properties | | | |
|---|---|---|---|---|---|
| | | Conversion, % | | Flex | Flex |
| | Viscosity, | Ambient | Post | strength, | modulus, |
| Comonomers | mPa · s | photocure | cure | MPa | GPa |

Mono-urethane monomethacrylates + UDMA and acidic comonomer

| | | | | | |
|---|---|---|---|---|---|
| UDMA/IEM-PhOH | 8804 ± 1185 | — | — | — | — |
| (70:30) | 119.7 ± 10.0 | 79.2 ± 4.7 | 88.5 ± 1.9 | 128.3 ± 14.9 | 3.42 ± 0.18 |
| ″ + MAA | | | | | |
| UDMA/PhNCO-HEMA | 1367 ± 107.0 | — | — | — | — |
| (70:30) | 63.6 ± 3.9 | 69.4 ± 1.6 | 89.3 ± 0.6 | 160.2 ± 26.3 | 4.44 ± 0.82 |
| ″ + MAA | | | | | |
| UDMA/IEM-BzOH | 999.6 ± 107.0 | — | — | — | — |
| (70:30) | 46.2 ± 2.2 | 72.5 ± 5.0 | 90.9 ± 0.9 | 184.5 ± 18.0 | 3.24 ± 0.30 |
| ″ + MAA | | | | | |
| UDMA/IEMEG-BzOH | 432.8 ± 32.1 | — | — | — | — |
| (70:30) | 41.9 ± 2.1 | 76.2 ± 2.5 | 95.0 ± 1.4 | 179.7 ± 28.3 | 4.01 ± 0.46 |
| ″ + MAA | | | | | |
| UDMA/IEM-BzOH | 191.1 ± 10.3 | 80.2 ± 1.6 | 97.6 ± 0.7 | 138.8 ± 12.2 | 3.99 ± 0.15 |
| (50:50) | 12.5 ± 0.7 | 74.3 ± 1.5 | 92.2 ± 0.9 | 154.5 ± 20.8 | 4.74 ± 0.29 |
| ″ + MAA | | | | | |
| UDMA/IEM-BuOH | 276.8 ± 20.7 | — | — | — | — |
| (70:30) | 26.7 ± 1.4 | 74.8 ± 2.2 | 92.6 ± 0.5 | 161.6 ± 16.9 | 3.99 ± 0.32 |
| ″ + MAA | | | | | |
| UDMA/IEM-IBOH | 1821 ± 181 | — | — | — | — |
| (70:30) | 77.6 ± 7.3 | 70.9 ± 5.6 | 87.8 ± 0.7 | 133.4 ± 22.2 | 2.91 ± 0.24 |
| ″ + MAA | | | | | |
| UDMA/IEM-PEOH | 1173 ± 61 | — | — | — | — |
| (70:30) | 46.5 ± 0.5 | 83.7 ± 6.4 | 91.6 ± 1.4 | 172.6 ± 26.8 | 3.90 ± 0.36 |
| ″ + MAA | | | | | |
| UDMA/IEM- | 1417 ± 234 | — | — | — | — |
| Triton45(70:30) | 110.3 ± 4.3 | 79.6 ± 2.0 | 93.3 ± 1.0 | 136.5 ± 22.6 | 3.44 ± 0.15 |
| ″ + MAA | | | | | |

Mono-urethane dimethacrylates + UDMA and acidic comonomer

| | | | | | |
|---|---|---|---|---|---|
| UDMA/IEM-HEMA | 188.5 ± 9.6 | 62.4 ± 1.7 | 85.5 ± 0.7 | 141.2 ± 18.6 | 3.63 ± 0.37 |
| (50:50) | 11.7 ± 0.9 | 64.1 ± 1.1 | 82.8 ± 1.1 | 191.6 ± 35.8 | 4.50 ± 0.33 |
| ″ + MAA | | | | | |
| UDMA/IEM-HPMA | 780.0 ± 43.6 | — | — | — | — |
| (70:30) | 53.3 ± 4.7 | 66.4 ± 4.2 | 83.5 ± 0.7 | 150.5 ± 21.9 | 2.79 ± 0.41 |
| ″ + MAA | | | | | |
| UDMA/IEM-HBMA | 397.9 ± 190.6 | — | — | — | — |
| (70:30) | 53.8 ± 2.3 | 66.6 ± 4.5 | 86.0 ± 1.1 | 174.6 ± 65.8 | 4.00 ± 1.42 |
| ″ + MAA | | | | | |
| UDMA/IEM-HBMA | 417.3 ± 136.7 | — | — | — | — |
| (70:30) | 53.7 ± 4.3 | 76.8 ± 4.0 | 90.4 ± 1.8 | 181.8 ± 22.1 | 4.19 ± 0.31 |
| ″ + AA/MAA (1:1) | | | | | |
| UDMA/IEM-HBMA | 348.9 ± 33.0 | — | — | — | — |
| (70:30) | 52.1 ± 7.4 | 83.8 ± 2.7 | 92.8 ± 0.7 | 171.4 ± 13.3 | 3.91 ± 0.22 |
| ″ + AA | | | | | |

As shown in Table 5, the viscosity and polymer properties are provided for resin formulations of UDMA with either mono-urethane monomethacrylates or mono-urethane dimethacrylates as a means to control both the pre-cure viscosity and the covalent network density in the copolymers without significantly reducing the overall urethane group concentration.

The mechanical properties of copolymers made with UDMA and 30 mol % of mono-urethane monomethacrylates as shown in Table 5, such as IEM-BzOH and IEM-PEOH, are comparable or better in terms of strength and modulus in the presence of MAA, relative to the UDMA homopolymer and the UDMA/MAA copolymer mechanical properties shown in Table 2.

Raising the mono-urethane monomethacrylate to 50 mol % dropped the strength but increased the modulus. With 50 mol % of the IEM-HEMA mono-urethane dimethacrylate used with UDMA, a very low viscosity resin is obtained that also produces improved polymeric strength and modulus results with MAA reinforcement, relative to the UDMA/MAA formulation.

One important aspect is that the commercially available diurethane dimethacylate UDMA can be leveraged here for a significant portion of the formulation that gives excellent polymeric mechanical properties while the blending of multi- and mono-urethane monomers provides resin viscosities well below the 100 mPa·s threshold, along with the ability to maintain a high overall urethane group content.

The enhancement in the ambient real-time conversion and post-cure conversion seen for the formulations that included AA as either the only acid-functional comonomer or as an equimolar mixture of AA and MAA with the acid to urethane group ratio fixed at 1:1, again demonstrates the ability of AA to facilitate the photopolymerization of urethane methacrylates, while maintaining the polymeric mechanical properties that are obtained with the MAA diluted and reinforced analog formulations. Also, the among the isomers of IEM-PhOH and PhNCO-HEMA that were both solids either with or without added MAA, these both mixed with UDMA to give homogeneous resins where the PhNCO-HEMA was significantly more effective in lowering the viscosity of UDMA and simultaneously providing good polymeric mechanical properties relative to the IEM-PhOH.

### Example 6. Composites

Composite compositions were prepared from moderate to low viscosity resin formulations according to the disclosure and an added filler. For the filler, a gamma-methacryloxypropyltrimethoxysilane surface treated barium glass filler of 0.7 μm mean particle size. Structures of tetraurethane monomers and anhydride comonomers (4-AETA, 4-META, 4-AMETA), used in the composite compositions are shown in FIG. 13. The composite formulations contained 2,2-dimethoxy-2-phenylacetophenone (DMPA; 0.1 wt %) and they were photocured for 20 seconds per side (top/bottom) with 365 nm light (filtered Hg arc lamp) at 100 mW/cm² intensity at ambient temperature. The composites were not subjected to post-curing unless noted otherwise.

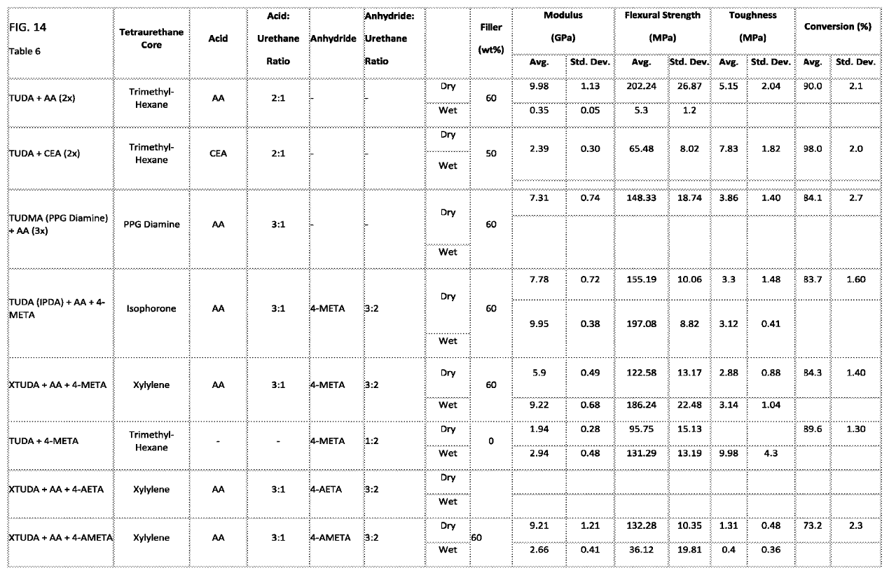

Composites were tested for Modulus (GPa), Flexural strength (MPa), and Toughness (MPa) physical properties under dry and wet conditions. Conversion (%) was also determined. Results are shown in Table 6. (FIG. 14).

Undesirable reductions in mechanical properties of both the unfilled and filled versions were observed when certain of the urethane-acid polymers-particularly some of the tetraurethane-based formulations-when exposed to water. This property decline was worse with acrylic acid vs methacrylic acid as the acid functional comonomer. In an attempt to remedy this water sensitivity, composite compositions were developed that incorporated both acidic and anhydride comonomers along with the tetraurethane monomers.

It was surprisingly found that incorporation of both acidic and anhydride comonomers along with the tetraurethane monomers yielded polymers that either tolerated water exposure better or actually significantly increased in both modulus and strength when fully water saturated vs dry.

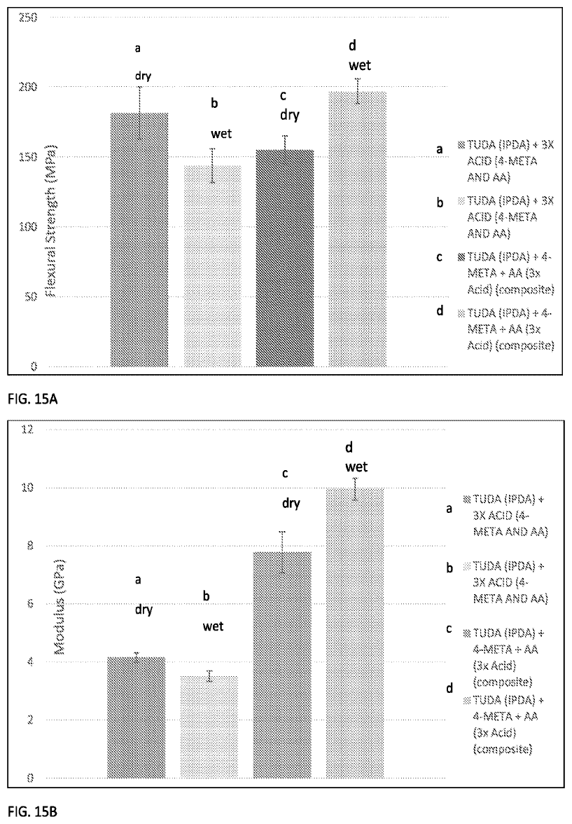

FIG. 15A shows a bar graph of flexural strength (MPa) unfilled and filled composite polymer compositions prepared from compositions comprising both acidic and anhydride comonomers along with the tetraurethane monomers [TUDA (IPDA)+3 acid (4-META and AA)]under wet and dry conditions. As shown in Table 6 (FIG. 14) the composition included an isophorone tetraurethane core, an acid: urethane ratio of 3:1, anhydride: urethane ratio of 3:2, and was either unfilled or included 60 wt % of the filler. The unfilled composition exhibited loss of flexural strength when wet (b) compared to dry conditions (a). In contrast, the filled composite composition exhibited increased flexural strength when wet (d) compared to under dry conditions (c).

FIG. 15B shows a bar graph of modulus (GPa) for unfilled and filled composite polymer compositions prepared from compositions comprising both acidic and anhydride comonomers along with the tetraurethane monomers [TUDA (IPDA)+3 acid (4-META and AA)]under wet and dry conditions. The unfilled composition exhibited some loss of modulus when wet (b) compared to dry conditions (a). In contrast, the filled composite composition exhibited substantially increased modulus when wet (d) compared to under dry conditions (c).

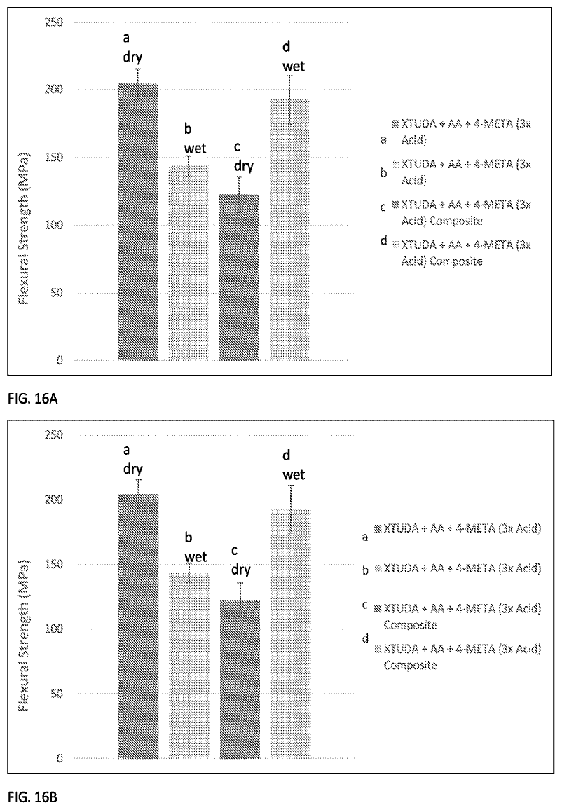

FIG. 16A shows a bar graph of flexural strength (MPa) unfilled and filled polymer composite compositions prepared from compositions comprising both acidic and anhydride comonomers along with the tetraurethane monomers [XTUDA+AA+4-META (3X acid)]under wet and dry conditions. As shown in Table 6 (FIG. 14) the composition included an xylylene tetraurethane core, a 3:1 acid: urethane ratio, a 3:2 anhydride: urethane ratio, and was either unfilled or included 60 wt % of the filler. The unfilled composition exhibited loss of flexural strength when wet (b) compared to dry conditions (a). In contrast, the filled composite composition exhibited increased flexural strength when wet (d) compared to under dry conditions (c).

FIG. 16B shows a bar graph of modulus (GPa) for unfilled and filled composite polymer compositions prepared from compositions comprising both acidic and anhydride comonomers along with the tetraurethane monomers [XTUDA+AA+4-META (3X acid)]under wet and dry conditions. The unfilled composition exhibited some loss of modulus when wet (b) compared to dry conditions (a). In contrast, the filled composite composition exhibited substantially increased modulus when wet (d) compared to under dry conditions (c).

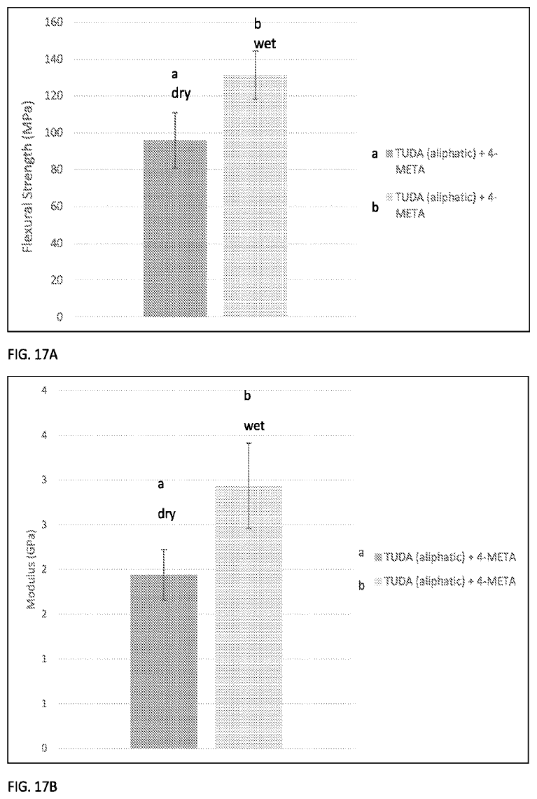

FIG. 17A shows a bar graph of flexural strength (MPa) of unfilled polymer composition prepared from compositions comprising a tetraurethane monomer and an anhydride comonomer without acid (TUDA+4-META) having an aliphatic trimethyl hexane tetraurethane core, and an anhydride: urethane ratio of 1:2. The unfilled composition exhibited increased flexural strength when wet (b) compared to dry conditions (a).

FIG. 17B shows a bar graph of modulus (GPa) of unfilled polymer composition prepared from compositions comprising a tetraurethane monomer and an anhydride comonomer without acid (TUDA+4-META) having an aliphatic trimethyl hexane tetraurethane core, and an anhydride: urethane ratio of 1:2. The unfilled composition exhibited increased modulus when wet (b) compared to dry conditions (a).

The above specification, examples and data provide a complete description of the manufacture and use of the composition of the invention. Since many embodiments of the invention can be made without departing from the spirit and scope of the invention, the invention resides in the claims hereinafter appended.

### REFERENCES

1. Maurya S D, Kurmvanshi S K, Mohanty S, Nayak S K. A Review on Acrylate-Terminated Urethane Oligomers and Polymers: Synthesis and Applications. Polymer-Plastics Technology and Engineering 2018; 57 (7): 625-56.
2. Lemon M T, Jones M S, Stansbury J W. Hydrogen bonding interactions in methacrylate monomers and polymers. Journal of Biomedical Materials Research Part A 2007; 83A (3): 734-46.
3. Decker C, Moussa K. REAL-TIME KINETIC-STUDY OF LASER-INDUCED POLYMERIZATION. Macromolecules 1989; 22 (12): 4455-62.
4. Decker C, Moussa K. PHOTOPOLYMERIZATION OF MULTIFUNCTIONAL MONOMERS.4. ACRYLATES WITH CARBAMATE OR OXAZOLIDONE STRUCTURE. European Polymer Journal 1991; 27 (4-5): 403-11.
5. Decker C, Moussa K. PHOTOPOLYMERIZATION OF MULTIFUNCTIONAL POLYMERS.5. POLYURETHANE-ACYLATE RESINS. European Polymer Journal 1991; 27 (9): 881-89.
6. Kilambi H, Cramer N B, Schneidewind L H, et al. Evaluation of highly reactive mono-methacrylates as reactive diluents for BisGMA-based dental composites. Dental Materials 2009; 25 (1): 33-38.
7. Kilambi H, Stansbury J W, Bowman C N. Enhanced reactivity of monovinyl acrylates characterized by secondary functionalities toward photopolymerization and Michael addition: Contribution of intramolecular effects. Journal of Polymer Science Part a-Polymer Chemistry 2008; 46 (10): 3452-58.
8. Kilambi H, Reddy S K, Schneidewind L, et al. Design, development, and evaluation of monovinyl acrylates characterized by secondary functionalities as reactive diluents to diacrylates. Macromolecules 2007; 40 (17): 6112-18.
9. Beckel E R, Stansbury J W, Bowman C N. Effect of aliphatic spacer substitution on the reactivity of phenyl carbamate acrylate monomers. Macromolecules 2005; 38 (8): 3093-98.
10. Lu H, Stansbury J W, Nie J, Berchtold K A, Bowman C N. Development of highly reactive mono-(meth)acrylates as reactive diluents for dimethacrylate-based dental resin systems. Biomaterials 2005; 26 (12): 1329-36.
11. Cramer N B, O'Brien C P, Bowman C N. Mechanisms, polymerization rate scaling, and oxygen inhibition with an ultra-rapid monovinyl urethane acrylate. Polymer 2008; 49 (22): 4756-61.
12. Berchtold K A, Nie J, Stansbury J W, et al. Novel monovinyl methacrylic monomers containing secondary functionality for ultrarapid polymerization: Steady-state evaluation. Macromolecules 2004; 37 (9): 3165-79.
13. Barner-Kowollik C, Bennet F, Schneider-Baumann M, et al. Detailed investigation of the propagation rate of urethane acrylates. Polymer Chemistry 2010; 1 (4): 470-79.
14. Meng L, Wang X J, Ocepek M, Soucek M D. A new class of non-isocyanate urethane methacrylates for the urethane latexes. Polymer 2017; 109:146-59.
15. Rokicki G, Parzuchowski P G, Mazurek M. Non-isocyanate polyurethanes: synthesis, properties, and applications. Polymers for Advanced Technologies 2015; 26 (7): 707-61.
16. Zareanshahraki F, Asemani H R, Skuza J, Mannari V. Synthesis of non-isocyanate polyurethanes and their application in radiation-curable aerospace coatings. Progress in Organic Coatings 2020; 138.
17. Tanaka J, Hashimoto T, Stansbury Jw, Antonucci J M, Suzuki K. Polymer properties on resins composed of UDMA and methacrylates with the carboxyl group. Dental Materials Journal 2001; 20:206-15.

1. A low viscosity polymerizable resin composition comprising a urethane (meth)acrylate monomer, and a comonomer selected from the group consisting of an acidic comonomer, an anhydride comonomer, and a monourethane mono(meth)acrylate comonomer, wherein the urethane (meth)acrylate monomer is selected from the group consisting of a multi-urethane (meth)acrylate monomer and a monourethane di(meth)acrylate comonomer.

2. The low viscosity polymerizable resin composition according to claim 1, wherein the multi-urethane (meth)acrylate monomer is selected from the group consisting of a tetraurethane di(meth)acrylate (TUD(M)A) monomer, a diurethane (meth)acrylate (DUM(M)A) monomer, hexaurethane tri(meth)acrylate (HUT(M)A) monomer, an octaurethane tetra(meth)acrylate (OUT(M)A) monomer, and a diurethane di(meth)acrylate monomer.

3. The low viscosity polymerizable resin composition according to claim 1, wherein the ratio of the acidic moieties from the acidic monomer to the urethane moieties from the urethane (meth)acrylate monomer are in an acidic moiety: urethane moiety ratio in a range of from about 1:1 to about 12:1, about 2:1 to about 8:1; or >1:1, about 2:1; about 3:1; about 4:1; about 5:1; about 6:1; about 7:1; about 8:1; about 9:1; or about 10:1.

4. The low viscosity polymerizable resin composition according to claim 2- or 3, wherein the TUD(M)A monomer comprises a chemical structure according to Formula (I): 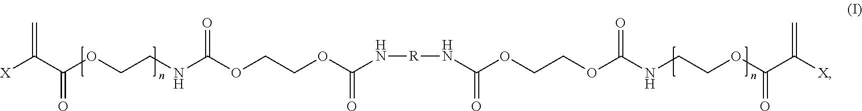
wherein X is —H or —CH₃; n is 1, 2, 3 or 4; and R is aliphatic, alkoxyalkyl, or alkylarylalkyl core group,
optionally wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group,
further optionally wherein n is 1 or 2; and R is a core radical selected from the group consisting of

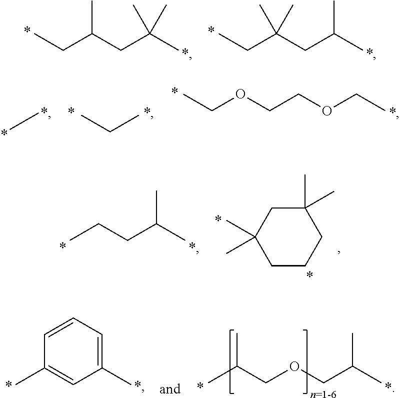

5. The low viscosity polymerizable resin composition according to claim 2, wherein the DUM(M)A monomer comprises a chemical structure according to Formula (IV): 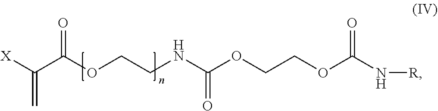
wherein X is —H or —CH₃; n is 1, 2, 3 or 4; and R is aliphatic, alkoxyalkyl, or alkylarylalkyl core group.

6. The low viscosity polymerizable resin composition according to claim 2, wherein the HUT(M)A monomer comprises a chemical structure according to Formula (V): 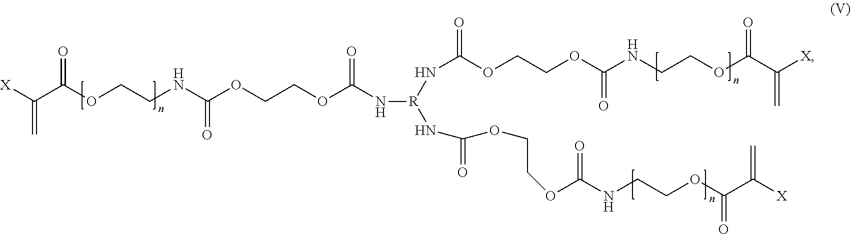
wherein X is —H or —CH₃; n is 1, 2, 3 or 4; and R is aliphatic, alkoxyalkyl, or alkylarylalkyl core group.

7. The low viscosity polymerizable resin composition according to claim 2, wherein the OUT(M)A monomer comprises a chemical structure according to Formula (VI): 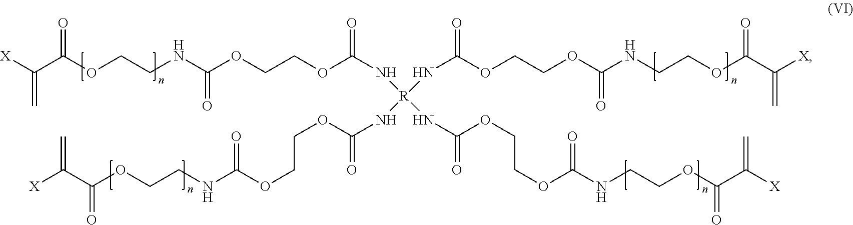
wherein X is —H or —CH₃; n is 1, 2, 3 or 4; and R is aliphatic, alkoxyalkyl, or alkylarylalkyl core group.

8. (canceled)

9. (canceled)

10. The low viscosity polymerizable composition according to claim 2, wherein the diurethane di(meth)acrylate monomer is selected from the group consisting of UD(M)A, IPDI-HE(M)A, a PCL diurethane di(meth)acrylate, HE(M)A-MDI, HE(M)A-IPDI, HE(M)A-TDI, HE(M)A-HMDI, HE(M)A-TMXDI, and HE(M)A-XDI.

11. The low viscosity polymerizable resin composition according to claim 1, wherein the acidic comonomer is selected from the group consisting of acrylic acid, methacrylic acid (MAA), itaconic acid, mono-2-(methacryloyloxy)ethyl maleate, pyromellitic dianhydride glycerol dimethacrylate, 2-carboxyethyl acrylate, 2-carboxyethyl acrylate oligomer, mono-2-(methacryloyloxy)ethyl succinate, glycerol dimethacrylate/succinate adduct, 1,3-glycerol dimethacrylate/maleate adduct, bis[2-(methacryloyloxy)ethyl]phosphate, or ethylene glycol methacrylate phosphate.

12. The low viscosity polymerizable resin composition according to claim 1, comprising a monourethane mono(meth)acrylate comonomer or a monourethane di(meth)acrylate comonomer.

13. The low viscosity polymerizable resin composition according to claim 1 comprising a diurethane di(meth)acrylate monomer, a monourethane mono(meth)acrylate comonomer or a monourethane di(meth)acrylate comonomer; and an acidic comonomer,
wherein the molar ratio of the diurethane di(meth)acrylate monomer to the monourethane mono(meth)acrylate monomer or the monourethane di(meth)acrylate monomer is from about 90:10 to about 50:50; about 80:20 to about 50:50; about 70:30, or about 50:50.

14. (canceled)

15. The low viscosity polymerizable resin composition according to claim 13, wherein the ratio of the acidic moieties from the acidic monomer to the urethane moieties from the combined urethane (meth)acrylate monomers are in an acidic moiety: urethane moiety ratio in a range of from about 1:1 to about 10:1, about 2:1 to about 8:1; or >1:1, about 2:1; about 3:1; about 4:1; about 5:1; about 6:1; about 7:1; about 8:1; about 9:1; or about 10:1.

16. The low viscosity polymerizable resin composition according to claim 1, wherein the monourethane mono(meth)acrylate monomer comprises a compound according to Formula (II): 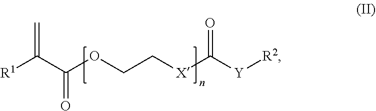
wherein n=1 or 2; R¹ is —H or —CH₃; X′ is O or N; Y is N when X′=O, or Y is O when X′ =N; R² is aliphatic, benzyl, alkoxyaryl, alkoxyalkyl, polyalkoxyalkyl, or optionally alkyl C₁₋₁₀ substituted polyalkoxyaryl, optionally
wherein the monourethane mono(meth)acrylate monomer is selected from the group consisting of: 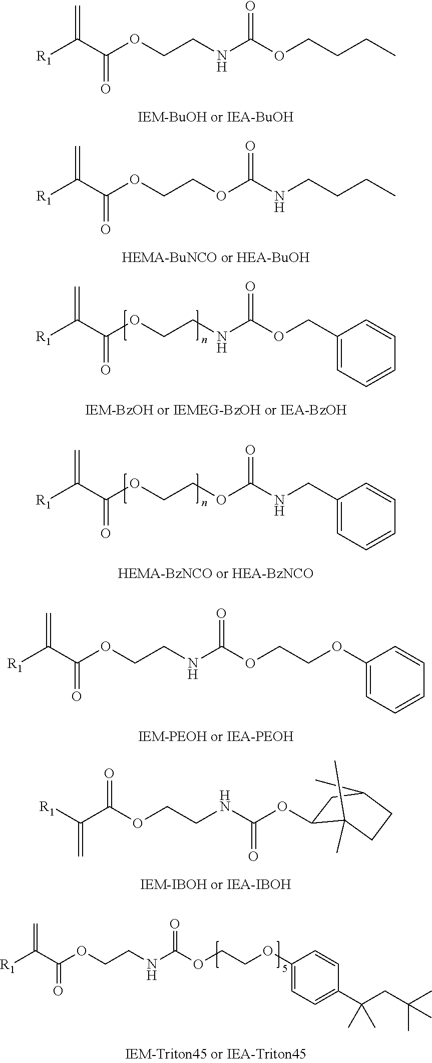
wherein R₁=H, or CH₃; n=1, 2, 3, 4, 5; n=1 for the IEM-BzOH monomer: n=2 for IEMEG-BzOH.

17. (canceled)

18. The low viscosity polymerizable resin composition according to claim 1, wherein the monourethane di(meth)acrylate comonomer is a compound according to Formula (IIIa): 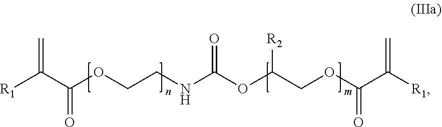
wherein R₁=—H, —CH₃; n=1 or 2; m=1-5; and R₂ is —H, aliphatic, aryl, or alkylaryl group; optionally wherein R2=H, Me, Et, nPr, iPr, nBu, sBu, tBu, Phe, or Bzl, optionally wherein the monourethane di(meth)acrylate comonomer is selected from the group consisting of 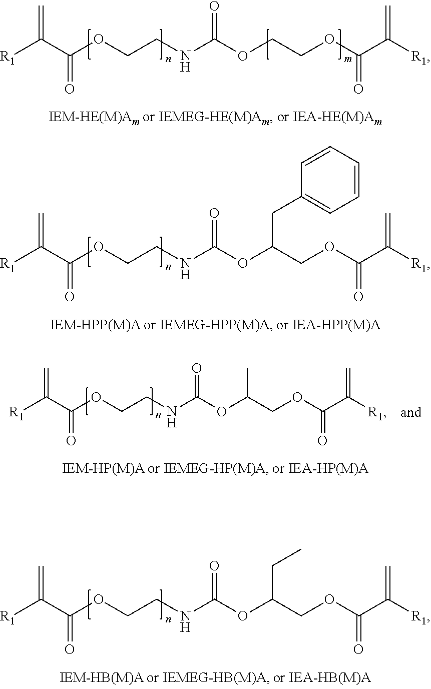
wherein R₁=CH₃ and n=1 for the IEM monomers, R₁=CH₃ and n=2 for the IEMEG monomers, and R₁=H and n=1 for the IEA monomers.

19. (canceled)

20. The low viscosity polymerizable resin composition claim 1, wherein the mono-urethane di(meth)acrylate comonomer is a compound according to Formula (IIIb): 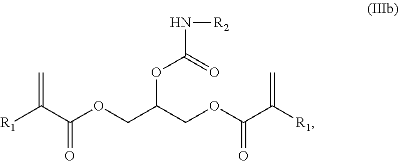
wherein R₁=—H, —CH₃; n=1 or 2; m=1-5, R₂=aliphatic, aryl, alkylaryl; optionally wherein R₂=Me, Et, nPr, iPr, nBu, sBu, tBu, Phe, or Bzl, optionally
wherein the mono-urethane di(meth)acrylate monomer is selected from the group consisting of

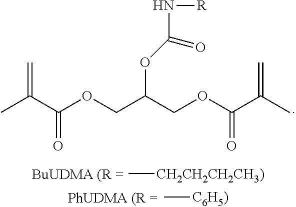

21. (canceled)

22. The low viscosity polymerizable resin composition according to claim 1, wherein the anhydride comonomer is selected from the group consisting of 4-AETA, 4-META, and 4-AMETA.

23. The low viscosity polymerizable resin composition according to claim 1, comprising a tetraurethane di(meth)acrylate monomer, an acidic comonomer, and an anhydride comonomer, optionally wherein the ratio of the anhydride moieties from the anhydride comonomer monomer to the urethane moieties from the multi-urethane (meth)acrylate monomer are in an anhydride moiety: urethane moiety ratio in a range of from about 5:1 to about 1:1, about 3:1 to about 1:1; or about 3:2.

24. (canceled)

25. The polymerizable resin composition according to claim 1, further comprising an initiator, optionally wherein the initiator is selected from the group consisting of a photoinitiator, a thermal initiator, and a redox initiator.

26. The polymerizable resin composition of claim 1, wherein the polymerizable resin is a low viscosity polymerizable resin having a viscosity at room temperature of no more than 1,000 mPa·s, no more than 500 mPa·s, no more than 300 mPa·s, 100 mPa·s, or no more than 80 mPa·s, no more than 60 mPa·s, no more than 40 mPa·s, or no more than 20 mPa·s.

27. A polymer prepared from the polymerizable resin according to claim 1, wherein the polymer exhibits:
flexural strength of at least 100 MPa, at least 130 MPa, at least 150 MPa, at least 180 MPa, or at least 200 MPa; and
flexural modulus of at least 2 GPa, at least 3 GPa, at least 4 GPa, or at least 5 GPa.

28. A method of preparing the polymer according to claim 27, comprising:
curing the polymerizable resin to form the polymer, wherein the curing comprises exposing the polymerizable resin to a condition for a period of time, wherein the condition is selected from the group consisting of light (photocuring), elevated temperature above ambient temperature (thermal curing), or redox conditions (redox curing); optionally wherein the light is selected from the group consisting of visible light and ultraviolet light, and wherein the photocuring is used to produce spatial and temporal on-demand polymer formation.

29. (canceled)

30. (canceled)

31. The method according to claim 28, wherein the polymer exhibits conversion at ambient temperature of at least 60%, or at least 70%, as determined by static or dynamic infrared spectroscopic methods or by another suitable analytical technique.

32. The method according to claim 28, further comprising:
post-curing the polymer, optionally
wherein the polymer exhibits post-curing conversion of at least 75%, at least 80%, at least 85%, or at least 90%.

33. (canceled)

34. A method for creating a two-dimensional film or a three-dimensional shaped part comprising molding, free-form fabricating, or printing of the polymerizable resin composition according to claim 1.

35. The method according to claim 34, comprising: three-dimensional (3D) printing of the polymerizable resin composition to form a partially or fully cured shaped part; and optionally subjecting to additional post-cure processing to complete production of the shaped part, optionally wherein the shaped part is a shaped dental appliance, dental prosthetic device, biomedical device, automotive part, microelectronics part, aerospace part, plumbing part, or electrical part.

36. (canceled)

37. A shaped part comprising: a polymer created from the polymerization of the polymerizable resin composition according to claim 1, optionally in admixture with one or more fillers.

38. A polymerizable dental appliance or prosthetic material comprising: the polymerizable resin composition of claim 1, and optionally fillers and/or pigments, optionally wherein the filler is present in a range of from 0-90 wt %, 0-50 wt %, or 0-25 wt % of the total material weight, and optionally a pigment in a range of from about 0.0001-5 wt %, 0.001-1 wt %, or 0.003-0.5 wt % of the total material weight.

39. (canceled)

40. A dispensing device comprising an unpolymerized quantity of the polymerizable dental appliance or prosthetic material of claim 38.

41. A tetraurethane di(meth)acrylate (TUD(M)A) monomer according to Formula (I) 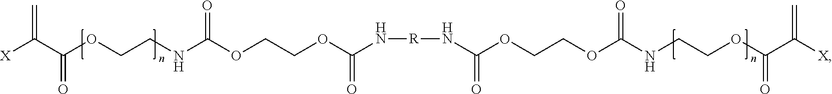
wherein X is —H or —CH₃; n is 1, 2, 3 or 4; and R is aliphatic, alkoxyalkyl, or alkylarylalkyl core group, optionally
wherein wherein X is —H or —CH₃; n is 1 or 2; and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group, further optionally wherein R is a core radical selected from the group consisting of

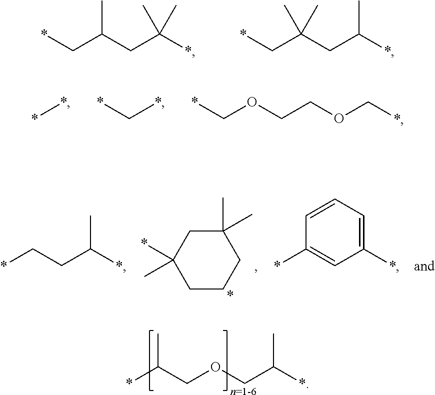

42. (canceled)

43. (canceled)

44. A monourethane mono(meth)acrylate monomer compound selected from the group consisting of: 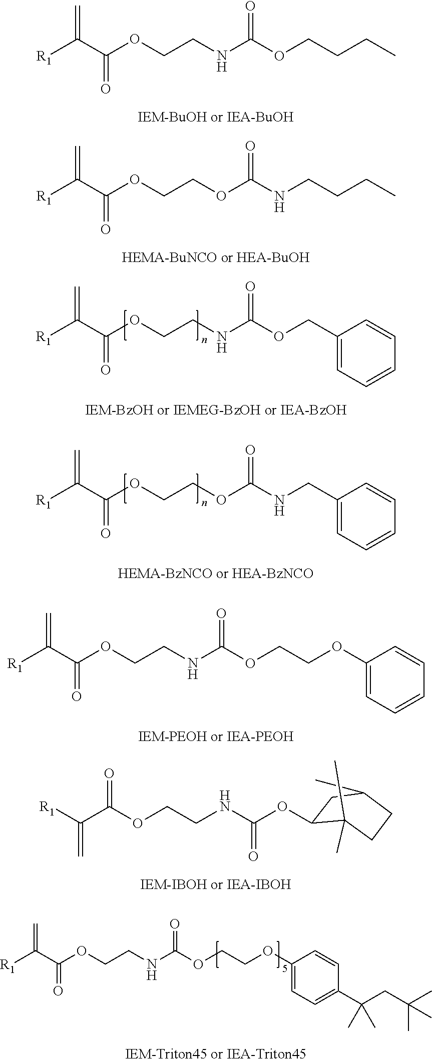
wherein R₁=H, or CH₃; n=1, 2, 3, 4, 5; n=1 for the IEM-BzOH monomer; n=2 for IEMEG-BzOH.

45. A mono-urethane di(meth)acrylate monomer compound according to Formula (IIIb): 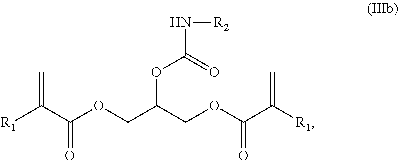
wherein R₁=—H, —CH₃; n=1 or 2; m=1-5, R₂=aliphatic, aryl, alkylaryl; optionally wherein R₂=Me, Et, nPr, iPr, nBu, sBu, tBu, Phe, or Bzl, optionally wherein the mono-urethane di(meth)acrylate monomer compound is selected from the group consisting of

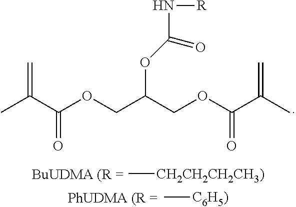

46. (canceled)

47. A diurethane (meth)acrylate (DUM(M)A) monomer comprising a chemical structure according to Formula (IV): 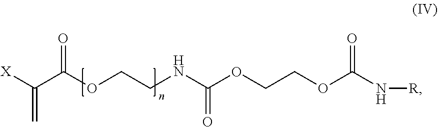
wherein X is —H or —CH₃; n is 1, 2, 3 or 4; and R is aliphatic, alkoxyalkyl, or alkylarylalkyl core group, optionally
wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

48. A hexaurethane tri(meth)acrylate (HUT(M)A) monomer comprising a chemical structure according to Formula (V): 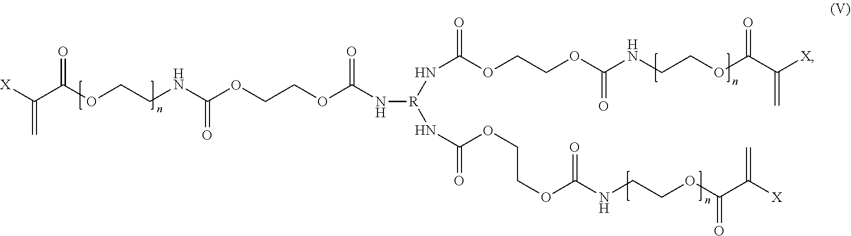
wherein X is —H or —CH₃; n is 1, 2, 3 or 4; and R is aliphatic, alkoxyalkyl, or alkylarylalkyl core group, optionally
wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

49. An octaurethane tetra(meth)acrylate (OUT(M)A) monomer comprising a chemical structure according to Formula (VI): 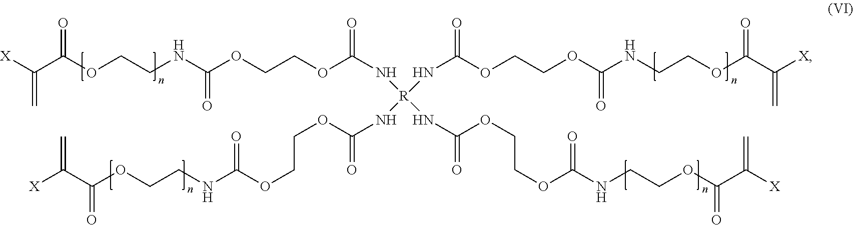
wherein X is —H or —CH₃; n is 1, 2, 3 or 4; and R is aliphatic, alkoxyalkyl, or alkylarylalkyl core group, optionally
wherein n=1 or 2, and R is a straight or branched chain alkyl C₂-C₂₀, 1,2-diethoxyethane, ethoxyethane, 1-ethoxy-2-(2-ethoxyethoxy) ethane, 1-propoxypropane, 1,3-xylenyl radical, or a 1,4-xylenyl radical core group.

50. (canceled)
